SEQUENCE LISTING (35 sequences)

SEQ ID NO: 1 - DNA, 6 bp, Artificial Sequence
  Synthetic construct
nggtgn

SEQ ID NO: 2 - DNA, 6 bp, Artificial Sequence
  Synthetic Construct
nngtnn

SEQ ID NO: 3 - DNA, 6 bp, Artificial Sequence
  Synthetic construct
nngtnn

SEQ ID NO: 4 - DNA, 3 bp, Artificial Sequence
  Synthetic construct
gng

SEQ ID NO: 5 - DNA, 9 bp, Artificial Sequence
  Synthetic construct
gggtggnnn

SEQ ID NO: 6 - DNA, 3 bp, Artificial Sequence
  Synthetic construct
gng

SEQ ID NO: 7 - DNA, 9 bp, Artificial Sequence
  Synthetic construct
gggtggnnn

SEQ ID NO: 8 - DNA, 3 bp, Artificial Sequence
  Synthetic construct
gng

SEQ ID NO: 9 - DNA, 9 bp, Artificial Sequence
  Synthetic construct
gggtggnnn

SEQ ID NO: 10 - DNA, 3 bp, Artificial Sequence
  Synthetic construct
ntn

SEQ ID NO: 11 - DNA, 6 bp, Artificial Sequence
  Synthetic construct
nngnnn

SEQ ID NO: 12 - DNA, 6 bp, Artificial Sequence
  Synthetic construct
nnnnnn

SEQ ID NO: 13 - DNA, 6 bp, Artificial Sequence
  Synthetic construct
nnntnn

SEQ ID NO: 14 - DNA, 6 bp, Artificial Sequence
  Synthetic construct
gggngg

SEQ ID NO: 15 - DNA, 6 bp, Artificial Sequence
  Synthetic construct
gggtng

SEQ ID NO: 16 - DNA, 6 bp, Artificial Sequence
  Synthetic construct
ggnngg

SEQ ID NO: 17 - DNA, 6 bp, Artificial Sequence
  Synthetic construct
ggntgg

SEQ ID NO: 18 - DNA, 6 bp, Artificial Sequence
  Synthetic construct
nggtgg

SEQ ID NO: 19 - DNA, 7 bp, Artificial Sequence
  Synthetic construct
gggtggn

SEQ ID NO: 20 - DNA, 7 bp, Artificial Sequence
  Synthetic construct
ngggtgg

SEQ ID NO: 21 - DNA, 6 bp, Artificial Sequence
  Synthetic construct
ggntgg

SEQ ID NO: 22 - DNA, 6 bp, Artificial Sequence
  Synthetic construct
gggngg

SEQ ID NO: 23 - DNA, 7 bp, Artificial Sequence
  Synthetic construct
ngggtgg

SEQ ID NO: 24 - DNA, 7 bp, Artificial Sequence
  Synthetic construct
gggtggn

SEQ ID NO: 25 - DNA, 8 bp, Artificial Sequence
  Synthetic construct
ngggtggn

SEQ ID NO: 26 - DNA, 6 bp, Artificial Sequence
  Synthetic construct
ggntgg

SEQ ID NO: 27 - DNA, 6 bp, Artificial Sequence
  Synthetic construct
gggngg

SEQ ID NO: 28 - DNA, 7 bp, Artificial Sequence
  Synthetic construct
ngggtgg

SEQ ID NO: 29 - DNA, 7 bp, Artificial Sequence
  Synthetic construct
gggtggn

SEQ ID NO: 30 - DNA, 8 bp, Artificial Sequence
  Synthetic construct
ngggtggn

SEQ ID NO: 31 - DNA, 22 bp, Artificial Sequence
  Synthetic construct
tgctgctttttgctggcttttt

SEQ ID NO: 32 - DNA, 24 bp, Artificial Sequence
  Synthetic construct
tcgtcgttttgtcgttttgtcgtt

SEQ ID NO: 33 - DNA, 21 bp, Artificial Sequence
  Synthetic construct
ggggacgacgtcgtcgggggg

SEQ ID NO: 34 - DNA, 24 bp, Artificial Sequence
  Synthetic construct
tcgtcgttttgtcgttttgtcgtt

SEQ ID NO: 35 - DNA, 22 bp, Artificial Sequence
  Synthetic construct
tcgtcgttttcggcggccgccg

(19) United States
(12) Patent Application Publication
(10) Pub. No.: US 2011/0111016 A1
(43) Pub. Date: May 12, 2011

(54) NON-DNA BASE-CONTAINING POLYNUCLEOTIDE COMPOSITIONS AND THEIR USE FOR THE MODULATION OF IMMUNE RESPONSES

(76) Inventors: Nigel C. Phillips, Point-Claire (CA); Mario C. Filion, Laval (CA)

(21) Appl. No.: 12/900,674

(22) Filed: Oct. 8, 2010

Related U.S. Application Data
(60) Provisional application No. 61/259,812, filed on Nov. 10, 2009.

(51) Int. Cl. A61K 9/127; A61K 31/7088; A61K 45/00; A61K 39/00; C07H 21/04; A61P 37/00

(52) U.S. Cl. 424/450; 514/44 R; 424/283.1; 424/184.1; 536/23.1

(57) ABSTRACT

The present invention provides compositions comprising synthetic non-DNA base-containing polynucleotide sequences of 3 to 30 bases in length comprising one or more non-DNA bases wherein the bases are nebularine, hypoxanthine, or uracil, or combinations of nebularine, hypoxanthine and uracil bases, in combination with a pharmaceutically acceptable vehicle, particularly one or more adjuvant vehicle, and one or more antigen. The present invention relates to methods of administering these compositions for inducing or modulating an immune response in vitro or in vivo, and particularly for activating antigen presenting cells.

### PRIOR RELATED APPLICATIONS

The present application claims the benefit of priority to U.S. Provisional Application No. 61/259,812 filed Nov. 10, 2009, which is hereby incorporated by reference in its entirety.

### FIELD OF THE INVENTION

The present invention relates to compositions comprising synthetic non-DNA base-containing polynucleotides, a pharmaceutically acceptable vehicle and optionally one or more antigens, and their use to modulate immune responses.

### BACKGROUND OF THE INVENTION

Appropriate recognition of microbial danger and cellular stress is vital to survival of the host as this leads to activation of local defense mechanisms and recruitment and activation of specialized immune cells. Thus, antigen presenting cells (APC), as well as other cells of the innate immune system, have evolved a variety of means for doing so, using so called “pattern recognition receptors” (PRRs). The PRRs recognize molecular patterns (pathogen-associated molecular patterns or PAMPs) in non-processed antigens such as cell wall components or nucleic acids of pathogens (bacterial, mycobacterial, viral) that are shared by large groups of microorganisms, but are distinct from those found in the host. APC may express PRRs including CD14, mannose receptor, DEC 205, and the family of toll-like receptors (TLRs). APCs comprise but are not necessarily limited to professional APC such as myeloid and plasmacytoid dendritic cells, monocytes, macrophages, Langerhans' cells, B lymphocytes, T lymphocytes, Kupffer cells, microglia, Schwann cells and endothelial cells; and non-professional APC such as, but not limited to, epithelial cells, fibroblasts, melanocytes, neural cells, smooth muscle cells, myocytes, hepatocytes, astrocytes and keratinocytes. In the prior art, substances typically of microbial origin or modifications thereof have been used to stimulate immune responses, often in a non-specific manner. Examples are the inclusion of mycobacteria in Freund's adjuvant, LPS, the combination of monophosphoryl lipid A (a less toxic derivative of LPS) and trehalose dimycolate (a mycolic acid species from mycobacteria), Poly(I:C), a synthetic analog of viral dsRNA, and palindromic DNA sequences containing CpG dinucleotide motifs. Several recent publications disclose the use of synthetic immunostimulatory polynucleotides containing an unmethylated CpG motif (CpGs). Two synthetic imidazoquinoline compounds (Resiquimod and Imiquimod) also have immunostimulatory activity. Toll-like receptors (TLR) are known to interact with a number of natural and synthetic molecules that can function as immune adjuvants. Examples of these molecules are Poly (I:C) polynucleotides, lipopolysaccharide and CpG-containing polynucleotides. As described above, the specific TLR used by a given compound have been identified (i.e., TLR9 recognizes CpGs).

Synthetic polynucleotides are polyanionic sequences. Synthetic polynucleotides are reported that bind selectively to nucleic acids, to specific cellular proteins, to specific nuclear proteins or to specific cell surface receptors. Synthetic phosphorothioate polynucleotides of 8 to 100 bases containing a least one unmethylated CpG dinucleotide have been shown to stimulate the immune system (U.S. Pat. No. 6,239,116). In particular, synthetic phosphorothioate polynucleotides containing a CpG motif (5′ purine-purine-Cytosine-Guanine-pyrimidine-pyrimidine-3′) have been found to stimulate the synthesis of cytokines such as IL-6, IL-12, IFN-gamma, TNF-alpha, and GM-CSF, the lytic activity of natural killer cells and the proliferation of B lymphocytes (Krieg, Annual Rev. Immunol. 2002, 20:709-760). Synthetic phosphorothioate polynucleotides including a CpG motif wherein the number of bases is greater than 14 have been reported to trigger maturation and activation of dendritic cells: increase in cell size and granularity; synthesis of IL-12; increase in endocytosis; and, up-regulation of cell surface molecules MHC II, CD40, CD80, CD83 and CD86 (Sparwasser et al. Eur. J. Immunol. 1998, 28:2045-205; Hartman et al. Proc. Natl. Acad. Sci. USA 1999, 96:9305-9310; Askew et al. J. Immunol. 2000, 165:6889-6895). Synthetic phosphodiester polynucleotides including a CpG motif wherein the number of bases is 30 have been reported to stimulate the synthesis of IFN and to up-regulate the expression of CD80 and CD86 on DC precursors (Kadowaski et al. J. Immunol. 2001 166:2291-2295).

We have previously described a composition comprising a 2 to 20 base 3′-OH, 5′-OH synthetic polynucleotide selected from the group consisting of (GₓT_{y})ₙ, (T_{y}Gₓ)ₙ, a(GₓT_{y})ₙ, a(T_{y}Gₓ)ₙ, (GₓT_{y})ₙb, (T_{y}Gₓ)ₙb, a(GₓT_{y})ₙb, and a(T_{y}Gₓ)ₙb, wherein x and y is an integer between 1 and 7, n is an integer between 1 and 12, a and b are one or more As, Cs, Gs or Ts, wherein the polynucleotides is between 2 and 20 bases. These compositions induce a response selected from the group consisting of induction of cell cycle arrest, inhibition of proliferation, induction of caspase activation, induction of apoptosis and stimulation of cytokine synthesis by monocytes and peripheral blood mononuclear cells (see PCT Publication No. WO 01/44465).

Accordingly, there is a need for compounds and related methods that induces maturation of APC for a more efficient immunogenic response in the absence of inappropriate cytokine induction. Such compounds should be capable of eliciting efficient and appropriate immune responses against antigens, especially under circumstances where antigen or antigens are in short supply and where the efficacy of a vaccine needs to be both effective and protective.

What are specifically needed are new non-DNA base-containing polynucleotide compositions and methods for using these compositions to modulate the function of immune cells, including APC, such that effective immune adjuvant responses are achieved.

### SUMMARY OF THE INVENTION

The present invention fulfills this need by providing compositions comprising a synthetic non-DNA base-containing polynucleotide of 3 to 30 bases in length comprising one or more non-DNA bases and a pharmaceutically acceptable vehicle, for example an adjuvant. Such polynucleotides are called non-DNA base-containing polynucleotides herein. In one embodiment, the present invention provides synthetic non-DNA base-containing polynucleotides of 3 to 20 bases in length comprising one or more non-DNA bases. In another embodiment, the present invention provides synthetic non-DNA base-containing polynucleotides of 3 to 9 bases in length comprising one or more non-DNA bases. Further, the present invention provides compositions comprising synthetic non-DNA base-containing polynucleotides of 3 to 30, 3 to 20, or 3 to 9 bases in length comprising one or more nebularine bases, one or more hypoxanthine bases, or one or more uracil bases, or combinations of nebularine, hypoxanthine and uracil bases. These polynucleotides optionally further comprise one or more guanine bases, or one or more thymine bases, or one or more adenine bases, or one or more cytosine bases, or combinations thereof.

The present invention provides novel compositions comprising one or more non-DNA base-containing polynucleotides and a pharmaceutically acceptable vehicle, for example an adjuvant, optionally combined with one or more antigens. These compositions are useful to modulate an immune response in vitro or in vivo. Such modulation may be an increase or a decrease in an immune response. Several of the compositions of the present invention permit lower doses of antigen to be used in a vaccine formulation as the one or more non-DNA base-containing polynucleotides in combination with a pharmaceutically acceptable vehicle, for example one or more adjuvants, and one or more antigens produce an efficacious immune response to a lower dose of antigen.

The present invention also provides methods for using these compositions by administering the composition in vitro or in vivo in order to induce an immune response. Further, the compositions of the present invention may be administered together with one or more therapeutic agent. Such administration of the compositions of the present invention may occur before, during or after administration of one or more therapeutic agents known to one of ordinary skill in the medical or veterinary arts. Any therapeutic agent known to one of ordinary skill in the medical or veterinary arts, and employed to treat diseases, may be used in combination with these compositions.

In one embodiment, the present invention provides a composition comprising synthetic 5′-OH, 3′-OH non-DNA base-containing polynucleotides of 3 to 30, 3 to 20, or 3 to 9 bases in length. In a further embodiment, these non-DNA base-containing polynucleotides comprise a phosphate backbone which is a phosphodiester or other suitable modification. Throughout the present application, the term non-DNA base-containing polynucleotides is understood to mean 5′-OH or 3′-OH polynucleotides.

In a further embodiment, modification of the 5′-OH or 3′-OH termini of the non-DNA base-containing polynucleotide may be made to confer protection, such as from intracellular or extracellular degradation. In one example, the 5′-OH or 3′-OH termini of the non-DNA base-containing polynucleotide may include, but is not limited to triethyleneglycol (TEG)-cholesteryl. Use may be made of additional nucleotides containing such bases including but not limited to adenine (A), guanine (G), cytosine (C), or thymine (T) that can be added to one or both termini such that the intrinsic activity of the molecule, i.e., it's immune adjuvant activity, is not materially affected. Use may be made of additional nucleotides containing additional non-DNA bases selected from nebularine (Neb), hypoxanthine (I) and uracil (U) that can be added to one or both termini such that the intrinsic activity of the molecule, i.e., it's immune adjuvant activity, is not materially affected.

In one embodiment, the phosphate backbone of the polynucleotide is a phosphodiester backbone. In another embodiment, the phosphate backbone of the polynucleotide is a modified phosphorothioate backbone. In other embodiments, the phosphate backbone of the polynucleotide is an amino backbone or comprises modifications to the ribose or 2′-deoxyribose moiety. Preferably, the 5′-OH, 3′-OH polynucleotides are synthetic and comprise non-DNA bases such as, but not limited to, nebularine (Neb), hypoxanthine (I) or uracil (U). It is well known in the art that the hypoxanthine base is referred to as inosine when present in a nucleotide (for example inosine monophosphate—IMP) and is defined herein as “I”. In one embodiment the non-DNA base-containing polynucleotides are selected from the group consisting of NebGGTGNeb (SEQ ID NO:1), UUGTUU (SEQ ID NO:2), IIGTII (SEQ ID NO:3), GNebG (SEQ ID NO:4), GGGTGGNebNebNeb (SEQ ID NO:5), GIG (SEQ ID NO:6), GGGTGGIII (SEQ ID NO:7), GUG (SEQ ID NO:8), and GGGTGGUUU (SEQ ID NO:9). The non-DNA base-containing polynucleotides produce an immune response in vitro, or when administered in vivo to a human or animal. The immune response may be systemic or local. In one embodiment, the non-DNA base-containing polynucleotides stimulate an APC in the human or animal to which the polynucleotides are administered. The non-DNA base-containing polynucleotides may also be administered to an APC directly in vitro for stimulation of the APC. The stimulated APC may then be administered to a human or an animal from which the APCs were derived for the activation of an immune response in the human or animal.

When administered to an APC, the non-DNA base-containing polynucleotides of the present invention may stimulate the APC by inducing a response, for example, selected from the group consisting of an increase in the production of IL-1β, IL-6, IL-12, MCP-1, RANTES, and other cytokines and/or chemokines and/or hematopoeitic growth factors by APC.

The present invention further provides a method of administering a composition comprising a non-DNA base-containing polynucleotide and a pharmaceutically acceptable vehicle to an animal or a human in an amount effective to induce stimulation of an immune response in the animal or human, and more preferably, the stimulation of one or more APC in the animal or human. In one embodiment, a preferred APC is a professional APC such as a dendritic cell. In another embodiment, one or more antigens are administered to the animal or human in addition to the composition comprising a non-DNA base-containing polynucleotide and a pharmaceutically acceptable vehicle, resulting in an antigen-specific immune response in the animal or human. Preferred antigens are tumor antigens, bacterial antigens, fungal antigens, viral antigens or self antigens such as but not limited to hepatitis surface antigen, H1N1 antigens in the form of inactivated or attenuated virions, Mycobacterium bovis strain bacillus Calmette Guérin (BCG) antigens in the form of intact mycobacteria or subunit antigens comprised of one or more antigenic determinants, parasitic antigens, plant antigens, or self-antigens. In some embodiments, stimulation of one or more APC results in a systemic immune response in the animal or human. In other embodiments, the immune response is local. The present invention also includes methods of administering a composition comprising a non-DNA base-containing polynucleotide and a pharmaceutically acceptable vehicle which may be an immunomodulatory agent, or modality, to an animal or a human in an amount effective to induce stimulation of an immune response in the animal or human.

The present invention also includes methods of administering a composition comprising one or more non-DNA base-containing polynucleotides and a reduced amount of one or more antigens in a pharmaceutically acceptable vehicle, optionally together with one or more adjuvants such that an effective immune response is achieved, thus optimizing an antigen-sparing strategy where antigens are in short-supply and may difficult and costly to prepare.

The present invention also provides a method comprising in vitro administration of a composition comprising a non-DNA base-containing polynucleotide and a pharmaceutically acceptable vehicle to APC, and more preferably, DC, containing one or more antigens, as recited above, wherein such administration results in the stimulation of the APC. The method may further include introduction of the stimulated APC into an animal or human from which the APCs were derived, for the stimulation of an immune response in the animal or human. The unexpected and surprising ability of these compositions comprising non-DNA base-containing polynucleotides and a pharmaceutically acceptable vehicle, optionally containing one or more antigens, to stimulate an immune response provides an important benefit for animals and humans.

The methods described herein may be used for treating diseases such as but not limited to cancer, for treating allergies, for vaccinating animals or humans against various pathogens, for treating autoimmune diseases and for preventing transplantation rejection. Accordingly, the invention is useful for the prevention and treatment of various diseases including but not limited to infectious disease, cancer, autoimmune disease, and acts of bioterrorism due to the deliberate dissemination of organisms such as viruses, bacteria and toxins, including but not limited to anthrax, botulism, plague, tularemia, smallpox and viral hemorrhagic fever. In some embodiments, the present invention achieves treatment of autoimmune diseases and prevention of transplantation rejection by increasing IL-12 synthesis. The synthesis of IL-12 can inhibit autoimmune diseases, transplantation rejection and graft-versus-host disease (GVHD), and allergies (See for example, Bagenstose et al., J. Immunol. 1998; 160:1612 (Downregulation of autoantibody production in autoimmunity by IL-12); Vogel et al., Eur. J. Immunol., 1996; 26:219 (Inhibition of B1 lymphocyte, a B lymphocyte subset implicated in the development of autoimmunity, by IL-12); Dey et al., Blood, 1998; 91:3315 (Inhibition of graft-versus-host disease (GVHD) by IL-12); Smits et al., Int. Arch Allergy Immunol., 2001; 126-102 (Modification of the pathogenic Th2 immune profile toward a Th1 profile by IL-12 in the treatment of allergies)). In other embodiments, the present invention achieves treatment of autoimmune diseases and prevention of transplantation rejection through vaccination (See for example, Zhang et al., J. Mol. Med. 1996; 74:653 (Vaccination against autoreactive B or T lymphocytes responsible for autoreactive diseases); Vignes et al., Eur. J. Immunol., 2000; 30:2460 (Vaccination against alloreactive T lymphocytes responsible for graft rejection); Liu et al., J. Exp. Med., 2002; 196:1013 (Induction of immune tolerance by delivery of dying cells to activated DC cells in situ)). Accordingly, in one embodiment, the present invention provides synthetic non-DNA base-containing polynucleotides of 3 to 30, 3 to 20, or 3 to 9 bases in length, comprising one or more nebularine bases, one or more hypoxanthine bases, or one or more uracil bases, or combinations of nebularine, hypoxanthine and uracil bases, in combination with a pharmaceutically acceptable vehicle. These sequences optionally further comprise one or more guanine bases or one or more thymine bases, or one or more adenine bases, or one or more cytosine bases, or combinations thereof. Preferably the polynucleotide composed of the nebularine, hypoxanthine, uracil, guanine, thymine, adenine and cytosine bases in the combinations described contains a phosphodiester backbone.

In another embodiment, the present invention provides a composition comprising one or more of the non-DNA base-containing polynucleotides in a pharmaceutically acceptable vehicle optionally in combination with one or more antigens and one or more adjuvants, and their use in a method effective to treat or prevent a disease in an animal, including a human.

In another embodiment, the present invention provides compositions and methods effective to vaccinate an animal or human. A benefit of these compositions and methods is that lower amounts of antigen may be employed to generate an efficacious immune response and provide protection.

In another embodiment, the present invention provides a composition and method effective to vaccinate an animal or human using antigen doses that are considered as being sub-optimal when used with conventional adjuvants.

In still another embodiment, the present invention provides a composition and method effective to treat or prevent disease in an animal or human.

In yet another embodiment, the present invention provides a composition and method effective to stimulate an immune response in an animal or human.

In another embodiment, the present invention provides a composition and method effective to induce maturation and/or activation of APC, and preferably, DC, in an animal or human.

In yet another embodiment, the present invention provides a composition and method effective to induce in vitro stimulation of APC containing an antigen for autologous administration of the stimulated APC to an animal or human.

In another embodiment, the present invention provides a composition and method effective to increase the production of IL-10, IL-6, IL-12, MCP-1, RANTES, and other cytokines and/or chemokines and/or hematopoietic growth factors by APC.

In still another embodiment, the present invention provides a composition and method that potentiates the effect of other therapeutic or immunomodulatory agents.

In another embodiment, the present invention provides a composition and method that potentiates the effect of one or more cytokines on APC, and preferably, DC.

These embodiments of the present invention will become apparent after a review of the following detailed description of the disclosed embodiment and the appended claims.

### DETAILED DESCRIPTION OF THE INVENTION

In one embodiment, the present invention provides new compositions of matter comprising one or more non-DNA base-containing polynucleotides and a pharmaceutically acceptable vehicle, which may be one or more adjuvants. In one embodiment, the present invention provides new compositions of matter comprising one or more non-DNA base-containing polynucleotides in a pharmaceutically acceptable vehicle, which may be an adjuvant, optionally combined with one or more antigens.

The present invention is useful for enhancing the immunological responses to vaccine antigens by acting as an APC stimulating adjuvant.

The present invention is useful for suppressing or reducing immunological responses by acting on APC.

In yet another embodiment, the present invention provides a method to stimulate an immune response in vitro or in vivo comprising administration of a composition comprising one or more non-DNA base-containing polynucleotides and a pharmaceutically acceptable vehicle or excipient, optionally combined with one or more antigens.

The term “antigen presenting cells (APC)” as used herein is defined as antigen-presenting cells in the body that are responsible for priming T cells to respond to a specific antigen, whereby the T cell further differentiates into an “effector” cell, which can have functions such a T helper cell or cytotoxic T cell or into a “memory” T cell. APC also secrete a variety of cytokines and chemokines, which stimulate and direct T cell function as well as stimulating other immune cells including innate immune system cells such as natural killer cells, which provide immediate, non-pathogen specific killing of pathogens. APC include, but are not limited to, monocytes, macrophages, epithelial cells, endothelial cells, B cells, microglia, granulocytic cells, plasmacytoid dendritic cells and myeloid or monocyte dendritic cells. The terms “dendritic cell” and “DC” include, but are not limited to, interstitial DC, Langerhans cell-derived DC, plasmacytoid DC and any progenitors of the aforementioned cells.

The present invention provides compositions comprising synthetic non-DNA base-containing polynucleotides and methods of using them. These synthetic polynucleotides of 3 to 9, 3 to 20, or 3 to 30 bases in length comprise one or more non-DNA bases comprising one or more nebularine bases, one or more hypoxanthine bases, or one or more uracil bases, or combinations of nebularine, hypoxanthine and uracil bases. In a preferred embodiment the novel synthetic polynucleotides are 3 to 9 bases in length. These sequences may optionally further comprise one or more of adenine bases, cytosine bases, guanine bases or thymine bases, or combinations thereof. Preferably the polynucleotides composed of the nebularine, hypoxanthine, uracil, guanine, thymine, adenine and cytosine bases in the combinations described contains a phosphodiester backbone. One or more of these sequences may be combined with an acceptable vehicle, such as a pharmaceutically acceptable vehicle, to form a composition. Further, these compositions may be combined with one or more antigens and optionally together with one or more adjuvants which may be pharmaceutically acceptable vehicles. These compositions may also be combined with one or more therapeutic agents known to one of ordinary skill in the art to be useful in generating a desired therapeutic response.

These compositions are useful in inducing an immune response. In one embodiment, a composition comprising a non-DNA base-containing polynucleotide and a pharmaceutically acceptable vehicle, optionally combined with one or more antigen, is administered to an animal or human, in an amount effective to induce an immune response in the animal or human. In one embodiment, a composition comprising a non-DNA base-containing polynucleotide and a pharmaceutically acceptable vehicle, optionally combined with one or more antigen, and one or more adjuvant, is administered to an animal or human, in an amount effective to induce an immune response in the animal or human.

The following notation is used to describe the sequence of bases in the polynucleotide sequences of the present invention: A=adenine; C=cytosine; G=guanine; I=hypoxanthine; Neb=nebularine; T=thymine; and, U=uracil. As used herein, a non-DNA base-containing polynucleotide sequence refers to a synthetic polynucleotide comprising at least one of the bases I, Neb or U, or combinations thereof, further optionally containing at least one of the bases A, C, G or T, or combinations thereof. The non-DNA base-containing polynucleotide sequence is 3 to 30, 3 to 30 or 3 to 9 bases in length.

The terms “a” or “an”, as used herein, are defined as one, or more than one. The terms “including” and/or “having”, as used herein, are defined as comprising (i.e., open language).

In a further embodiment, the present invention provides a composition comprising a synthetic non-DNA base-containing polynucleotide sequence comprising 3 to 9, 3 to 20, or 3 to 30 bases, wherein at least one of the bases is a non-DNA base. In another embodiment, the non-DNA base-containing polynucleotide contains at least two non-DNA bases. The present invention also provides a method including administration of a composition comprising a non-DNA base-containing polynucleotide and a pharmaceutically acceptable vehicle to an animal or a human in an amount effective to stimulate one or more antigen-presenting cells (APC), or preferably one or more DC, in the animal or human. In a preferred embodiment, the non-DNA base-containing polynucleotide is a 5′-OH, 3′-OH polynucleotide. The stimulation of one or more APC in the animal or human may result in a systemic immune response or a local immune response. The present invention also provides a method including in vitro administration of a composition comprising a non-DNA base-containing polynucleotide to an APC containing an antigen in an amount effective to stimulate the APC. These APC's may then be administered in an autologous manner to an animal or human with a pharmaceutically acceptable vehicle for stimulation of an immune response. In a preferred embodiment, the non-DNA base-containing polynucleotide administered is a 5′-OH, 3′-OH polynucleotide. The unexpected and surprising ability of a non-DNA base-containing synthetic phosphodiester and phosphorothioate polynucleotides to induce stimulation of APC provides an important benefit for animals and humans.

Throughout the present application, the non-DNA base-containing polynucleotides are understood to mean 5′-OH, 3′-OH polynucleotides. As used herein the term “5′-OH, 3′-OH non-DNA base-containing polynucleotide” refers to a polynucleotide having hydroxyl moieties at both its 5′ and 3′ ends and comprising one or more non-DNA bases. More particularly, the 5′-OH, 3′-OH non-DNA base-containing polynucleotides of the present invention comprise a hydroxyl moiety at the 5′ carbon of the sugar at the 5′ end of the polynucleotide and comprise a hydroxyl moiety at the 3′ carbon of the sugar at the 3′ end of the polynucleotide. Unless otherwise indicated, the polynucleotide sequences described herein are shown in orientation from the 5′ terminus to the 3′ terminus, from left to right.

However, it is understood that one of ordinary skill in the art can modify one or both of the 5′-OH or 3′-OH termini with additions such as, but not limited to, TEG-cholesteryl, providing that the modification to the termini does not materially affect the intrinsic immune adjuvant activity of the polynucleotide. One or more additional A, T, C, or G bases may be added to either the 5′-OH terminus or 3′-OH terminus provided that the modification to the termini does not materially affect the intrinsic immune adjuvant activity of the polynucleotide. One or more additional nucleotides containing additional non-DNA bases selected from nebularine, hypoxanthine and uracil can be added to one or both termini such that the intrinsic activity of the molecule, i.e., it's immune adjuvant activity, is not materially affected.

As used herein the term “polynucleotide” and “oligonucleotide” are interchangeable. In one embodiment of the present invention, the non-DNA base-containing polynucleotide is 3 to 9 nucleotide bases in length. In another embodiment of the present invention, the non-DNA base-containing polynucleotide is 3 to 20 nucleotide bases in length. In yet another embodiment of the present invention, the non-DNA base-containing polynucleotide is 3 to 30 nucleotide bases in length. Preferably, the 5′-OH, 3′-OH non-DNA base-containing polynucleotide comprises nucleotide bases wherein at least one of those nucleotide bases is a non-DNA base such as, but not limited to, uracil (U), nebularine (Neb), or hypoxanthine (I). In some embodiments, the non-DNA base-containing polynucleotide may specifically comprise or consist of a base sequence selected from the group consisting of NebGGTGNeb (SEQ ID NO:1), UUGTUU (SEQ ID NO:2), IIGTII (SEQ ID NO:3).

In another embodiment, wherein the non-DNA base-containing polynucleotide comprises nebularine, the polynucleotide sequence can comprise any of the following sequences: NebTNeb (SEQ ID NO:10), GNebG (SEQ ID NO:4), NebNebGNebNebNeb (SEQ ID NO:11), NebNebNebNebNebNeb (SEQ ID NO:12), NebNebNebTNebNeb (SEQ ID NO:13), GGGNebGG (SEQ ID NO:14), GGGTNebG (SEQ ID NO:15), GGNebNebGG (SEQ ID NO:16), GGNebTGG (SEQ ID NO:17), NebGGTGG (SEQ ID NO:18), NebGGTGNeb (SEQ ID NO:1), GGGTGGNeb (SEQ ID NO:19) and NebGGGTGG (SEQ ID NO:20).

In another embodiment, wherein the non-DNA base-containing polynucleotide comprises hypoxanthine, the polynucleotide sequence can comprise any of the following sequences: GGITGG (SEQ ID NO:21), GGGIGG (SEQ ID NO:22), IIGTII (SEQ ID NO:3), IGGGTGG (SEQ ID NO:23), GGGTGGI (SEQ ID NO:24), IGGGTGGI (SEQ ID NO:25), GGGTGGIII (SEQ ID NO:7) and GIG (SEQ ID NO:6).

In another embodiment, wherein the non-DNA base-containing polynucleotide comprises uracil, the polynucleotide sequence can comprise any of the following sequences: GGUTGG (SEQ ID NO:26), GGGUGG (SEQ ID NO:27), UUGTUU (SEQ ID NO:2), UGGGTGG (SEQ ID NO:28), GGGTGGU (SEQ ID NO:29), UGGGTGGU (SEQ ID NO:30), GGGTGGUUU (SEQ ID NO:9) and GUG(SEQ ID NO:8).

In one embodiment, the non-DNA base-containing polynucleotides are administered to an APC, a DC, a human, or an animal in a nucleic acid-based vector.

The non-DNA base-containing polynucleotides may contain a phosphodiester backbone or other suitable modifications.

As also used herein, the terms “stimulate” and “stimulates” refer to the activation or maturation of an APC, or to the activation or increase of an immune response, depending upon the context of the terms' use.

It is believed that the non-DNA base-containing polynucleotides described herein are not only able to stimulate DC, but are also able to stimulate other APC including professional APC such as, but not limited to, monocytes, macrophages, Langerhans' cells, B lymphocytes, T lymphocytes, Kupffer cells, microglia, Schwann cells and endothelial cells; and non-professional APC such as, but not limited to, epithelial cells, fibroblasts, melanocytes, neural cells, smooth muscle cells, myocytes, hepatocytes, astrocytes and keratinocytes. Accordingly, the present invention includes compositions

When referring to an immune response, the term “stimulate” refers to an activation of the immune system generally or to an activation of components of the immune system in an antigen non-specific manner unless otherwise indicated. Stimulation of an immune response in an individual may be evidenced by, but is not limited to, cellular proliferation, clonal expansion, synthesis of new proteins, differentiation into effector cells, differentiation into memory cells, an increase in the level or amount of a type of antibody, a switch in the antibody class, somatic hypermutation in antibody-producing B lymphocytes, an increase in the level or amount of a type of immune cell, recruitment (motility and migration) of immune cells in a particular location, an increase in the level or amount of a cytokine in an individual, an increase in the level or amount of a chemokine in an individual, increased antigen presentation, increased endocytosis, an increase or acquisition of co-stimulatory molecules (accessory molecules), an increase or acquisition of adhesion molecules, an increase or acquisition of cytokine receptors, an increase or acquisition of chemokine receptors, increased cell-mediated cytotoxicity, morphological changes, establishment of immune cell memory, an increase in the level or amount of reactive oxygen intermediates, an increase in the level or amount of nitric oxide, an increase in the level or amount of neuroendocrine molecules (e.g., hormones, neurotransmitters, etc.), and a break of immune tolerance or suppression. Immune cells include, but are not limited to, lymphocytes such as B cells, T cells, including CD4⁺ and CD8⁺ cells, and NK cells; mononuclear phagocytes; granulocytes such as neutrophils, eosinophils and basophils; dendritic cells as described herein; and any progenitors of the aforementioned cells. Antibody types include IgG, IgA, IgM, IgD, IgE and IgY. IgG antibodies can be further divided into the different isotypes. In humans these are IgG1, IgG2, IgG3 and IgG4. In mice they are IgG1, IgG2a, IgG2b and IgG3. Antiviral immune responses are known to be associated with specific IgG antibody isotype responses. In mice these are known to those of ordinary skill in the art to be the IgG2a and IgG2b isotypes. The induction of antibodies of this class by viral vaccines is considered by those of ordinary skill in the art to be a measure of viral vaccine efficacy and potency.

In one embodiment, the immune response is a systemic immune response. The term “systemic immune response” refers herein to an immune response that is not restricted to a particular area of the body. An example of a systemic immune response is an increase in the level of an antibody circulating in the circulatory or lymphatic system in an individual following administration of an antigen and an immunostimulatory molecule to the individual. Another example is an increase in the level of a cytokine and/or a chemokine in the circulatory or lymphatic system in an individual following administration of an immunostimulatory molecule to the individual. Another example is the presence of sensitized immune effector cells such as T-cells, B-cells or plasma cells capable of responding to challenge with sensitizing antigen in the blood or lymphatic circulation or in immune system organs such as the spleen, lymph nodes or liver. In other embodiments, the immune response is a local immune response. The term “local immune response” refers an immune response that is primarily, but not necessarily wholly, restricted to a particular area of the body. A local immune response may be evidenced by localized swelling or redness and/or recruitment (motility and migration) of immune cells to a particular area of the body. For example, a mucosal immune response may occur following mucosal administration of an antigen and/or an immunostimulatory molecule such as a 5′-OH 3′-OH non-DNA base-containing polynucleotide sequence described herein. A mucosal response may include, but are not limited to, an increase in the level or amount of a type of antibody, an increase in the level or amount of IgA antibody, activation of gamma/delta-positive T lymphocytes, induction of local immune tolerance and induction of systemic immune tolerance.

In several embodiments of the present invention, the compositions comprising non-DNA base-containing polynucleotides are administered to an individual (i.e., an animal or a human) for the treatment or prevention of a disease. As used herein, the term “disease” refers to a condition wherein bodily health is impaired and includes, but is not limited to, a cancer; an infection by a pathogen including a virus, bacteria or parasite; an allergy; an autoimmune disease; and an autoimmune response to a transplanted organ. In some embodiments, the disease is associated with DC malfunction including, but not limited to, Sezary syndrome (patients have a profound defect in circulating DC) (Wysocka et al., Blood 2002, 100:3287); Down's syndrome (patients have a dendritic atrophy) (Takashima et al., J. Intellect. Disabil. Res. 1994, 38:265); autoimmune diseases involving inappropriate activation of DC (e.g., prolonged presentation of self antigen by DC) (Erikson et al., J. Exp. Med. 2003, 197:323 and Ludewig et al., Curr. Opin. Immunol. 2001, 13:657); spinal cord injury (patients have a dendritic atrophy) (Iversen et al., Blood 2000, 96:2081); and Graves' disease (thyroidal dendritic cells are implicated in the disease) (Quadbeck et al., Scand. J. Immunol. 2002, 55:612). The term “treatment” refers to the lessening or reduction of a disease symptom and does not require curing of the disease. As also used herein, the term “effective amount” refers to an amount of a non-DNA base-containing polynucleotide effective to induce an immune response. The therapeutic effectiveness of a non-DNA base-containing polynucleotide may be increased by methods including, but not limited to, chemically modifying the base, sugar or phosphate backbone to increase stability in biological milieu, chemically supplementing the oligonucleotide to enhance interactions with immune system cells or biotechnologically amplifying the sequences using bacterial plasmids containing the appropriate sequences to increase the availability of the sequences in vivo, complexing the sequences to biological or chemical carriers or coupling the sequences to cell-type directed ligands or antibodies. The term “effective amount” as used herein means an amount that is determined by such considerations as are known in the art of treating secondary immunodeficiencies wherein it must be effective to provide measurable relief in treated individuals, such as exhibiting improvements including, but not limited to, improved survival rate, more rapid recovery, improvement or elimination of symptoms, reduction of post infectious complications and, where appropriate, antibody titer or increased titer against the infectious agent, reduction in tumor mass, and other measurements as known to those skilled in the art.

The term “antigen” as used herein is defined as any material that can be specifically bound by an antibody, T-cell receptors, or pattern recognition receptors (PRRs), thereby inducing an immune response. Types of antigens include, but are not limited to viral, bacterial, tumor and self-antigens. Accordingly, the dendritic cells prepared according to this invention are useful for the prevention and treatment of various diseases including infectious disease, cancer, autoimmune disease, and due to the deliberate dissemination of organisms such as viruses, bacteria and toxins, including but not limited to anthrax, botulism, plague, tularemia, smallpox and viral hemorrhagic fever.

It is to be understood that compositions comprising one or more 5′-OH, 3′-OH non-DNA base-containing polynucleotides, a pharmaceutically acceptable vehicle and optionally one or more antigens may be administered in order to either stimulate antibody production for the production of monoclonal and polyclonal antibody production for applications such as, but not limited to, therapeutic treatment of diseases or in diagnostic purposes, for imaging or other applications known to those of skill in the art

It is to be understood that compositions comprising one or more 5′-OH, 3′-OH non-DNA base-containing polynucleotides, a pharmaceutically acceptable vehicle and optionally one or more antigens may be administered as a means of reducing the antibody response against allergens, desensitization against allergens, or as a means of reducing autoantibody responses in diseases such as but not limited to Hashimoto's disease, systemic lupus erythematosus and reactive arthritis.

It is to be understood that compositions comprising one or more 5′-OH, 3′-OH non-DNA base-containing polynucleotides may be administered in a pharmaceutically acceptable vehicle to an APC in vitro either alone, or in combination with other immunomodulatory agents, one or more antigens or one or more adjuvants that affect APC containing antigens, including tumor antigens, in a amount effective to induce stimulation of APC designed to be re-injected in an autologous manner to an animal or human, for the stimulation of the immune response. Immunomodulatory agents include, but are not limited to the following: aluminum hydroxide; aluminum phosphate; calcium phosphate; polymers; co-polymers such as polyoxyethylene-polyoxypropylene copolymers, including block co-polymers; polymer P1005; Freund's complete adjuvant (for animals); Freund's incomplete adjuvant; sorbitan monooleate; squalene; CRL-8300 adjuvant; QS 21; saponins; ISCOM; muramyl dipeptide; glucosaminylmuramyl dipeptide; trehalose; bacterial extracts, including mycobacterial extracts; bacterial whole cells, including mycobacterial whole cells; mycobacterial cell wall-DNA complex, mycobacterial cell wall skeletons, detoxified endotoxins; membrane lipids; DNA isolated from prokaryotic organisms, CpG synthetic polynucleotides; modified CpG synthetic polynucleotides or non-CpG containing polynucleotides (e.g. MIMPs), apatamers; plasmids immunostimulatory molecules; poly (I:C) molecules; cytokines; chemokines; chitosan and derivatives; hyaluronic acid and derivatives; cholera toxin; pertussis toxin; and, keyhole limpet hemocyanin, or combinations thereof. The compositions comprising one or more 5′-OH, 3′-OH non-DNA base-containing polynucleotide or 5′-OH, 3′-OH non-DNA base-containing polynucleotides plus immunomodulatory agent can be added to an APC in a single treatment or in multiple treatments, optionally at different concentrations, and over a period of time appropriate for the stimulation of an APC. The compositions comprising one or more 5′-OH, 3′-OH non-DNA base-containing polynucleotide can be administered before, at the same time, or after administration of the immunomodulatory agents. Moreover, compositions comprising one or more 5′-OH, 3′-OH non-DNA base-containing polynucleotide can be added before, at the same time, or after administration of the antigen(s).

The present invention also includes methods of administering a composition comprising a non-DNA base-containing polynucleotide and a pharmaceutically acceptable vehicle and an antigen directly as a vaccine to an animal or human, wherein such administration results in an immune response in the animal or human, and more preferably, an antigen-specific immune response. The antigen may be administered to the animal or human prior to, at the same time or following the administration of the composition comprising one or more non-DNA base-containing polynucleotides and a pharmaceutically acceptable vehicle. In a preferred embodiment, the antigen is administered at the same time as the composition comprising one or more 5′-OH, 3′-OH non-DNA base-containing polynucleotides and a pharmaceutically acceptable vehicle.

The present invention also includes methods of administering a composition comprising a non-DNA base-containing polynucleotide and a pharmaceutically acceptable vehicle, and an antigen not contained within an APC to an animal or human, wherein the amount of antigen is significantly decreased and such administration results in an effective immune response in the animal or human, and more preferably, an antigen-specific immune response. The antigen may be administered to the animal or human prior to, at the same time or following the administration of the composition comprising the non-DNA base-containing polynucleotide and a pharmaceutically acceptable vehicle. In a preferred embodiment, the antigen is administered at the same time as the composition comprising the non-DNA base-containing polynucleotide. In one embodiment, the antigen may be combined with the composition comprising a non-DNA base-containing polynucleotide and a pharmaceutically acceptable vehicle and administered to the animal or the human.

The antigens described herein are not limited to any particular antigen or type of antigen. Indeed it is to be appreciated that one of ordinary skill in the art after reading the detailed description and examples of the present invention would be lead to use the polynucleotide compositions of the present invention with antigens other than described in the present invention without departing from the spirit and the scope of the present invention as defined in the detailed description, examples and claims. Any antigen or more than one antigen may be selected by one of ordinary skill in the art for combination with the non-DNA base-containing polynucleotides of the present invention and one or more pharmaceutically acceptable vehicles in a composition. Such antigens are specific for the desired immune response or vaccine against a particular antigen. In one embodiment, the antigen is a tumor antigen, such as a tumor antigen derived from a tumor cell lysate or from techniques known to those of ordinary skill in the field of molecular biology (such as but not limited to sequencing, cloning, transfection, amplification in appropriate prokaryotic or eukaryotic cell-based systems and subsequent isolation and purification). In another embodiment, the antigen is an antigen derived from a pathogen, and more preferably an antigen expressed on the outer surface of the pathogen. One example of a pathogen derived surface antigen is the hepatitis surface antigen. In another embodiment the antigen is composed of an intact pathogen. One example of an intact pathogen is the inactivated or attenuated H1N1 virion. Another example is intact Mycobacterium bovis strain bacillus Calmette-Guérin (BCG), either unmodified or genetically modified where appropriate to express antigens protective against other mycobacterial infections such as but not limited to M. tuberculosis or Mycobacterium avium and sub-species such as paratuberculosis. When the antigen is administered to the animal or human not contained within an APC, the antigen may be administered as a protein, peptide or polypeptide, or a polynucleotide encoding the antigen may be administered. The polynucleotide encoding the antigen may be contained within a vector comprising other elements that will allow for expression of the antigen polypeptide in the animal or human. In one embodiment, the antigen and the 5′-OH, 3′-OH non-DNA base-containing polynucleotide are contained within different vectors. Antigens include but are not limited to H1N1 antigens, influenza antigens, hepatitis A antigens, hepatitis B antigens, hepatitis A antigens and hepatitis B antigens combined, human papilloma antigens, pneumococcal antigens, diphtheria antigens, meningococcal antigens, tetanus antigens, one or more human immunodeficiency virus antigens, one or more feline immunodeficiency virus antigens, and one or more simian immunodeficiency virus antigens

It is to be understood that the administration of a non-DNA base-containing polynucleotide-stimulated APC containing an antigen, or administration of a composition comprising a non-DNA base-containing polynucleotide, a pharmaceutically acceptable vehicle and antigen or antigens, to an animal or to a human, results in the stimulation of an immune response in the animal or human. In preferred embodiments, such administrations result in stimulation of the immune system in conjunction with an antigen-specific immune response in the animal or human. The term “antigen-specific immune response” refers to an immune response that is predominantly directed toward the antigen or antigens. An antigen-specific immune response includes or consists of but is not limited to, an increase in the amount of an antibody (antibody titer), a switch in the antibody class, somatic hypermutation in antibody-producing B lymphocytes, establishment of immune cell memory, increase in the amount of cells bearing a specific B cell receptor or T cell receptor for the antigen in the human or animal to which the antigen is administered. An antibody is “specific for” a particular antigen when the antibody binds to the antigen with sufficient affinity and avidity to result in the production of an antibody-antigen complex.

The compositions of the present invention comprising one or more non-DNA base-containing polynucleotides in a pharmaceutically acceptable vehicle, optionally combined with one or more antigens, include, but are not limited to, means or methods of administration such as injections, solutions, creams, gels, implants, pumps, ointments, emulsions, suspensions, microspheres, particles, microparticles, nanoparticles, liposomes, pastes, patches, tablets, transdermal delivery devices, sprays, aerosols, or other means familiar to one of ordinary skill in the art. Pharmaceutical formulations of the present invention can be prepared by procedures known in the art using well-known and readily available ingredients. For example, the compositions of the present invention can be formulated with common excipients, diluents, or carriers, and formed into tablets, capsules, suspensions, powders, and the like. Pharmaceutically acceptable vehicles are known to one of ordinary skill in the art, and include common excipients, diluents, or carriers. For example, the compositions can be formulated with common excipients, diluents, or carriers, and formed into tablets, capsules, suspensions, powders, and the like. Examples of excipients, diluents, and carriers that are suitable for such formulations include the following: fillers and extenders (e.g., starch, sugars, mannitol, and silicic derivatives); binding agents (e.g., carboxymethyl cellulose and other cellulose derivatives, alginates, gelatin, and polyvinyl-pyrrolidone); moisturizing agents (e.g., glycerol); disintegrating agents (e.g., calcium carbonate and sodium bicarbonate); agents for retarding dissolution (e.g., paraffin); resorption accelerators (e.g., quaternary ammonium compounds); surface active agents (e.g., cetyl alcohol, glycerol monostearate); adsorptive carriers (e.g., kaolin and bentonite); emulsifiers; preservatives; sweeteners; stabilizers; coloring agents; perfuming agents; flavoring agents; lubricants (e.g., talc, calcium and magnesium stearate); solid polyethyl glycols; and mixtures thereof. Pharmaceutically acceptable vehicles include pharmaceutically acceptable carriers. Some carriers may be immune adjuvant carriers known to one of ordinary skill in the art.

The formulations can be so constituted that they release the active ingredient only or preferably in a particular location, possibly over a period of time (i.e., a sustained-release formulation). Such combinations provide yet a further mechanism for controlling release kinetics. The coatings, envelopes, and protective matrices may be made, for example, from polymeric substances or waxes.

Compositions comprising one or more 5′-OH, 3′-OH non-DNA base-containing polynucleotides and a pharmaceutically acceptable vehicle are prepared by uniformly and intimately bringing into association the one or more 5′-OH, 3′-OH non-DNA base-containing polynucleotide and the pharmaceutically acceptable vehicle. Pharmaceutically acceptable vehicles may be liquid vehicles, solid vehicles or both. Liquid vehicles are aqueous vehicles, non-aqueous vehicles or both. Pharmaceutically acceptable vehicles include immune adjuvant carriers. Pharmaceutically acceptable vehicles include, but are not limited to, aqueous suspensions, oil emulsions, water-in-oil emulsions, water-in-oil-in-water emulsions, site-specific emulsions, long-residence emulsions, sticky-emulsions, microemulsions and nanoemulsions. Solid vehicles are biological vehicles, chemical vehicles or both and include, but are not limited to, viral vector systems, particles, microparticles, nanoparticles, microspheres, nanospheres, minipumps, bacterial cell wall extracts and biodegradable or non-biodegradable natural or synthetic polymers that allow for sustained release of the non-DNA base-containing polynucleotide compositions. Emulsions, minipumps and polymers can be implanted in the vicinity of where delivery is required (Brem et al., 1991 J. Neurosurg. 74: 441). Methods used to complex 5′-OH, 3′-OH non-DNA base-containing polynucleotide to a solid vehicle include, but are not limited to, direct adsorption to the surface of the solid vehicle, covalent coupling to the surface of the solid vehicle, either directly or via a linking moiety, and covalent coupling to the polymer used to make the solid vehicle. Optionally, a sequence(s) can be stabilized by the addition of non-ionic or ionic polymers such as polyoxyethylenesorbitan monooleates (commonly termed TWEENs) or hyaluronic acid or hyaluronic acid derivatives.

Preferred aqueous vehicle include, but are not limited to, water, saline and pharmaceutically acceptable buffers. Preferred non-aqueous vehicle include, but are not limited to, a mineral oil or a neutral oil including, but not limited to, a diglyceride, a triglyceride, a phospholipid, a lipid, an oil and mixtures thereof, wherein the oil contains an appropriate mix of polyunsaturated and saturated fatty acids. Examples include, but are not limited to, soybean oil, canola oil, palm oil, olive oil and myglyol, wherein the fatty acids can be saturated or unsaturated. Optionally, excipients may be included regardless of the pharmaceutically acceptable vehicle used to present the non-DNA base-containing polynucleotide compositions to cells. These excipients include, but are not limited to, anti-oxidants, buffers, and bacteriostats, and may include suspending agents and thickening agents.

One or more non-DNA base-containing polynucleotides may be administered to a human or an animal alone, or in combination with other immune adjuvant vehicles or carriers including, but not limited to, the following: alum, aluminum hydroxide; aluminum phosphate; calcium phosphate; polymers; co-polymers such as polyoxyethylene-polyoxypropylene copolymers, including block co-polymers; polymer P1005; Freund's complete adjuvant (for animals); Freund's incomplete adjuvant; sorbitan monooleate; squalene; squalane; CRL-8300 adjuvant; QS 21; saponins; ISCOM; muramyl dipeptide; glucosaminylmuramyl dipeptide; trehalose; hydrophilic or lipophilic bacterial extracts, including mycobacterial extracts; bacterial whole cells, including mycobacterial whole cells; bacterial cell wall preparations, including mycobacterial cell wall preparations; detoxified endotoxins; lipid A and synthetic analogs thereof; membrane lipids; DNA isolated from prokaryotic organisms, CpG synthetic polynucleotides; non-CpG synthetic polynucleotides; apatamers; plasmids encoding immunostimulatory molecules; poly (I:C) molecules; cytokines; chemokines; chitosan and derivatives; hyaluronic acid and derivatives; cholera toxin; pertussis toxin and keyhole limpet hemocyanin or combinations thereof. Immune adjuvant vehicles or carriers are known to one of ordinary skill in the art, and include, without limitation, alum, oil-based adjuvants, immunostimulating complexes (ISCOMS), virosomes, monophosphoryl lipid A (MPL), and/or analogs thereof. In a preferred embodiment of the present invention, the particulate carrier is alum, a colloidal antigen carrier which is prepared from either aluminum hydroxide or aluminum phosphate and which is used as a vaccine adjuvant.

Compositions comprising one or more non-DNA base-containing polynucleotides in a pharmaceutically acceptable vehicle may be administered alone, or in combination with other therapeutic modalities known to one of ordinary skill in the art, including, but not limited to, chemotherapeutic agents, antimicrobial agents, or antiviral agents. Chemotherapeutic agents include, but are not limited to, anti-metabolites, DNA damaging, microtubule destabilizing, microtubule stabilizing, actin depolymerizing, growth inhibiting, topoisomerase inhibiting, HMG-CoA inhibiting, purine inhibiting, pyrimidine inhibiting, metalloproteinase inhibiting, CDK inhibiting, angiogenesis inhibiting and differentiation enhancing. Dosages and methods of administration of these other therapeutic modalities are known to one of ordinary skill in the art.

Methods of administering the compositions comprising the non-DNA base-containing polynucleotide compositions of the present invention and a pharmaceutically acceptable vehicle, APC or DC containing the non-DNA base-containing polynucleotides, or compositions comprising non-DNA base-containing polynucleotides and other materials such as carriers of the present invention, that are particularly suitable for various forms include, but are not limited to the following types of administration, oral (e.g., buccal or sublingual), anal, rectal, as a suppository, topical, parenteral, nasal, aerosol, inhalation, intrathecal, intraperitoneal, intravenous, intraarterial, transdermal, intradermal, subdermal, subcutaneous, intramuscular, intratissular (e.g., tissue or gland), intrauterine, vaginal, into a body cavity, surgical administration at the location of a tumor or internal injury, directly into tumors, into the lumen or parenchyma of an organ, into bone marrow and into any mucosal surface of the gastrointestinal, reproductive, urinary and genitourinary system. The compositions comprising the 5′-OH, 3′-OH non-DNA base-containing polynucleotides of the present invention and a pharmaceutically acceptable vehicle can also be administered to a mucosal surface selected from the group consisting of intravesical (bladder), ocular, oral, nasal, rectal and vaginal surface. Techniques useful in the various forms of administrations mentioned above include but are not limited to, topical application, ingestion, surgical administration, injections, sprays, transdermal delivery devices, osmotic pumps, electrodepositing directly on a desired site, or other means familiar to one of ordinary skill in the art. Sites of application can be external, such as on the epidermis, or internal, for example a gastric ulcer, a surgical field, or elsewhere.

The compositions of the present invention can be applied in the form of creams, gels, solutions, suspensions, liposomes, particles, or other means known to one of skill in the art of formulation and delivery of the compositions. Ultrafine particle sizes can be used for inhalation delivery of therapeutics. Some examples of appropriate formulations for subcutaneous administration include, but are not limited to, implants, depot, needles, capsules, and osmotic pumps. Some examples of appropriate formulations for vaginal administration include but are not limited to creams and rings. Some examples of appropriate formulations for oral administration include but are not limited to: pills, liquids, syrups, and suspensions. Some examples of appropriate formulations for transdermal administration include but are not limited to gels, creams, pastes, patches, sprays, and gels. Some examples of appropriate delivery mechanisms for subcutaneous administration include, but are not limited to, implants, depots, needles, capsules, and osmotic pumps. Formulations suitable for parenteral administration include, but are not limited to, aqueous and non-aqueous sterile injection solutions which may contain anti-oxidants, buffers, bacteriostats and solutes which render the formulation isotonic with the blood of the intended recipient, and aqueous and non-aqueous sterile suspensions which may include suspending agents and thickening agents. Extemporaneous injection solutions and suspensions may be prepared from sterile powders, granules and tablets commonly used by one of ordinary skill in the art.

Embodiments in which the compositions of the invention are combined with, for example, one or more pharmaceutically acceptable vehicles or excipients may conveniently be presented in unit dosage form and may be prepared by conventional pharmaceutical techniques. Such techniques include the step of bringing into association the compositions containing the active ingredient and the pharmaceutical vehicle(s) or excipient(s). In general, the formulations are prepared by uniformly and intimately bringing into association the active ingredient with liquid vehicles. Preferred unit dosage formulations are those containing a dose or unit, or an appropriate fraction thereof, of the administered ingredient. It should be understood that in addition to the ingredients particularly mentioned above, formulations comprising the compositions of the present invention may include other agents commonly used by one of ordinary skill in the art. The volume of administration will vary depending on the route of administration.

Such volumes are known to one of ordinary skill in the art of administering compositions to animals or humans. Depending on the route of administration, the volume per dose is preferably about 0.001 to 100 mL per dose, more preferably about 0.01 to 50 mL per dose and most preferably about 0.1 to 30 mL per dose. For example, intramuscular injections may range in volume from about 0.1 ml to 1.0 ml. The non-DNA base-containing polynucleotide compositions administered alone, or together with other therapeutic agent(s), can be administered in a single dose treatment, in multiple dose treatments, or continuously infused on a schedule and over a period of time appropriate to the disease being treated, the condition of the recipient and the route of administration. Moreover, the other therapeutic agent can be administered before, at the same time as, or after administration of the compositions comprising one or more non-DNA base-containing polynucleotides.

Preferably, the amount of 5′-OH, 3′-OH non-DNA base-containing polynucleotide administered per dose is from about 0.0001 to 100 mg/kg body weight, more preferably from about 0.001 to 10 mg/kg body weight and most preferably from about 0.01 to 5 mg/kg body weight. The particular non-DNA base-containing polynucleotide and the particular therapeutic agent administered, the amount per dose, the dose schedule and the route of administration should be decided by the practitioner using methods known to those skilled in the art and will depend on the type of disease, the severity of the disease, the location of the disease and other clinical factors such as the size, weight and physical condition of the recipient. In addition, in vitro assays may optionally be employed to help identify optimal ranges for sequence and for sequence plus therapeutic agent administration.

For use in the present invention, non-DNA base-containing polynucleotides can be synthesized de novo using any of a number of procedures well known in the art. For example, the β-cyanoethyl phosphoramidite method (Beaucage and Caruthers 1981 Tet. Let. 22:1859) and the nucleoside H-phosphonate method (Garegg et al. 1986 Tet. Let. 27:4051-4054; Froehler et al. 1986 Nucl. Acid. Res. 14:5399-5407; Garegg et al. 1986 Tet. Let. 27:4055-4058, Gaffney et al. 1988 Tet. Let. 29; 2619-26220), or by the use of liquid-phase synthetic procedures (Bonora et al. 1993 Nucl. Acids Res. 21:1213-1217) can be utilized. These chemistries can be performed by a variety of automated oligonucleotide synthesizers available in the market. Alternatively, polynucleotides can be prepared from existing nucleic acid sequences (e.g. genomic or cDNA) using known techniques, such as those employing restriction enzymes, exonucleases, and/or endonucleases.

For use in vivo, polynucleotides are preferably relatively resistant to degradation (e.g. via endo- and exo-nucleases). One example of polynucleotide stabilization is via the use of phosphate backbone modifications. In one embodiment, a stabilized polynucleotide has a phosphorothioate-modified backbone. The phosphorothioate backbone modification of a polynucleotide results in a systemic half-life of forty-eight hours in rodents and such pharmacokinetics have resulted in the suggestion that they may be useful for in vivo applications (Agrawal et al. 1991 Proc. Natl. Acad. Sci. USA 88:7595). Phosphorothioates may be synthesized using automated techniques employing either phosphoramidate or H phosphonate chemistries. Aryl- and alkyl-phosphonates can be made (for example as described in U.S. Pat. No. 4,469,863); and alkylphosphotriesters (in which the charged oxygen moiety is alkylated as described in U.S. Pat. No. 5,023,243 and in European Patent No. 092,574) can be prepared by automated solid phase synthesis using commercially available reagents. Methods for making other DNA backbone modifications and substitutions have been described (Uhlmann, E. and Peyman, A. (1990) Chem. Rev. 90:544; Goodchild, J. (1990) Bioconjugate Chem. 1:165).

For administration in vivo, compositions comprising one or more non-DNA base-containing polynucleotides can be associated with a molecule that results in higher affinity binding to target cell (e.g., B-cell and natural killer (NK) cell) surfaces and/or increased cellular uptake by target cells. Polynucleotides can be ionically, or covalently associated with appropriate molecules using techniques, which are well known in the art. A variety of coupling or cross-linking agents can be used (e.g., protein A, carbodiimide, and N-succinimidyl-3-(2-pyridyldithio) propionate (SPDP)). Polynucleotides can alternatively be encapsulated in liposomes or virosomes using well-known techniques.

The following examples will serve to further illustrate the present invention without, at the same time, however, constituting any limitation thereof. On the contrary, it is to be clearly understood that resort may be had to various other embodiments, modifications, and equivalents thereof which, after reading the description herein, may suggest themselves to those skilled in the art without departing from the spirit and scope of the invention.

### Example 1

### Preparation of 5′-OH, 3′-OH non-DNA base-containing polynucleotides

The following 5′-OH, 3′-OH non-DNA base-containing polynucleotide sequences were prepared by Sigma-Genosys (Oakville, ON, Canada) or Midland (Midland, Tex., USA). Unless stated otherwise, the sequences were dispersed in autoclaved deionized water or in a pharmaceutically acceptable buffer such as, but not limited to, saline immediately prior to use.

It will be apparent to one of ordinary skill in the art that non-DNA base-containing polynucleotides may be prepared using routine or standard phosphoroamidite oligonucleotide technology.

The following 5′-OH, 3′-OH non-DNA base-containing polynucleotide sequences are only representative of compositions that can be used as an immune adjuvant. It is to be expected that modifications to these molecules such as, but not limited to, those described below, can be used by one of ordinary skill in the art of vaccine adjuvant formulation. It is clear from the following examples however that the general principle of using polynucleotide sequences containing Neb, U or I to act as immune adjuvants will be preserved. Similarly, the choice of an appropriate adjuvant formulation known to one skilled in the art will also preserve the general principle of using polynucleotide sequences containing Neb, U or I to act as immune adjuvants.

Additionally, use may be made of appropriate synthesis methods to generate polynucleotide sequences with appropriate backbone modifications, or other chemical modifications known to those of skill in the art that confer enhanced resistance to extracellular and intracellular degradation. Use may also be made of modification at the 5′- or 3′-termini of the ODN to confer protection (such as but not limited to TEG-cholesteryl addition or to backbone protection such as but not limited to the use of phosphorothioate linkages). Use may also be made of additional natural or non-natural nucleotides containing bases such as, but limited to, A, G, C, T, Neb, U or I that are added to one or both termini of the polynucleotide such that the intrinsic immune adjuvant activity is not materially affected. Use may be made of ribose and/or deoxyribose sugars or modifications without detracting from the scope and spirit of the invention.

The following sequences were synthesized with a phosphodiester backbone using standard phosphoroamidite polynucleotide technology:

| | |
|---|---|
| (SEQ ID NO: 1) | NebGGTGNeb |
| (SEQ ID NO: 2) | UUGTUU |
| (SEQ ID NO: 3) | IIGTII |
| (SEQ ID NO: 4) | GNebG |
| (SEQ ID NO: 5) | GGGTGGNebNebNeb |
| (SEQ ID NO: 6) | GIG |
| (SEQ ID NO: 7) | GGGTGGIII |
| (SEQ ID NO: 8) | GUG |
| (SEQ ID NO: 9) | GGGTGGUUU |

These molecules serve to illustrate the general composition of non-DNA base-containing polynucleotides that can be used as immune adjuvants, either as a simple mixture with the antigen or antigens against which an immune response is desired, or as a complex mixture or formulation with a carrier and one or more additional adjuvants designed to optimize an immune response. Such approaches commonly use a carrier molecule or material such as alum (aluminum phosphate or hydroxide), but the choice of carriers or vehicles available to those of skill in the art is broad and one of ordinary skill in the art would recognize them as being applicable to the polynucleotide sequences of the present invention without departing from the spirit and the scope of the present invention as defined in the following examples and claims.

The following reference polynucleotides were also synthesized with a phosphorothioate backbone and were purchased from Midland (Midland, Tex., USA):

(SEQ ID NO: 31) Non-CpG 4010
5′-TGCTGCTTTTTGCTGGCTTTTT-3′
(SEQ ID NO: 32) CpG 2006
5′-TCGTCGTTTTGTCGTTTTGTCGTT-3′,
(A-class)
(SEQ ID NO: 33) CpG 2336
5′-GGGGACGACGTCGTCGGGGGG-3′
(A-class)
(SEQ ID NO: 34) CpG 7909
5′-TCGTCGTTTTGTCGTTTTGTCGTT-3′
(B-class)
(SEQ ID NO: 35) CpG 2429
5′-TCGTCGTTTTCGGCGGCCGCCG-3′
(C-class)

### Example 2

### Lack of TLR Activation by Non-DNA Base-Containing Polynucleotides

Toll-like receptors (TLR) are known to interact with a number of natural and synthetic molecules that can function as immune adjuvants. Examples of these molecules are Poly (I:C) polynucleotides, lipopolysaccharide and CpG-containing polynucleotides. The interaction of non-DNA base-containing polynucleotides (SEQ ID NOs: 1-3) to interact with TLR and induce down-stream signaling was tested by assessing NF-κB activation in HEK293 cells expressing human TLR2, 3, 4, 5, 7, 8 and 9. TLR engagement was determined by measurement of secreted alkaline phosphatase, under the control of a reporter inducible by NF-κB. Appropriate positive controls were used for each TLR. The non-DNA base-containing polynucleotides were dissolved in sterile water at a concentration of 100 μg/mL, and used at a final concentration of 10 μg/mL. HEK293 cells containing the appropriate TLR and NF-κB reporting system (InvivoGen, San Diego, Calif., USA) were placed in wells of 96 well plates (25,000-50,000 cells, in a final volume of 200 μL medium designed for the detection of NF-κB induced secreted alkaline phosphatase expression), and incubated with non-DNA base-containing polynucleotides or positive controls for 16-20 hours. TLR activating activity was determined by measurement of absorbance at 650 nm wavelength using a Coulter AD340C absorbance detector. The results shown are expressed as Optical Density (OD). The results of a representative assay are shown in Table 1.

TABLE 1
TLR activation by non-DNA base-containing polynucleotides.

| HEK293/TLR | No | SEQ ID | SEQ ID | SEQ ID | Positive |
|---|---|---|---|---|---|
| cell line | ligand | NO: 1 (Neb) | NO: 2 (U) | NO: 3 (I) | control² |
| hTLR2 | 0.097¹ | 0.093 | 0.095 | 0.095 | 3.485 |
| hTLR3 | 0.055 | 0.055 | 0.056 | 0.057 | 2.547 |
| hTLR4 | 0.054 | 0.050 | 0.052 | 0.050 | 2.144 |
| hTLR5 | 0.129 | 0.158 | 0.143 | 0.155 | 2.593 |
| hHTR7 | 0.082 | 0.097 | 0.090 | 0.093 | 2.190 |
| hTLR8 | 0.125 | 0.118 | 0.120 | 0.128 | 3.137 |
| hTLR9 | 0.089 | 0.074 | 0.090 | 0.083 | 1.904 |
| NF-κB | 0.071 | 0.078 | 0.076 | 0.074 | 2.765 |
| control cells | | | | | |

¹Optical Density at 650 nm
²Positive controls:
hTLR2: heat killed Listeria monocytogenes at 10⁸ cells/mL
hTLR3: Poly (I:C) at 1 μg/mL
hTLR4: E. coli K12 LPS at 100 ng/mL
hTLR5: S. typhimurium flagellin at 100 ng/mL
hTLR7: Gardiquimod at 1 μg/mL
hTLR8: CL075 at 1 μg/mL
hTLR9: CpG ODN 2006 at 100 ng/mL
NK-κB control cells: PMA at 100 ng/mL

The results demonstrate that non-DNA base-containing polynucleotides are not capable of activating TLR-mediated signaling pathways, as evidenced by the lack of alkaline phosphates reporter activity (Table 1). In contrast, each TLR receptor was activated by its specific ligand (see the footnote to Table 1 for the respective positive control ligand used). While not wishing to be held to the following statement, these results support the hypothesis that non-DNA base-containing polynucleotides immune adjuvant activity is not mediated via TLR's, but by one or more different pathways.

Additionally, these results demonstrate that non-DNA base-containing polynucleotides do not signal through the known nucleic acid TLR receptors 3, 7, 8 or 9. Importantly, the results demonstrate that non-DNA base-containing polynucleotides are distinct from CpG-containing polynucleotides, both compositionally and mechanistically, as evidenced by the inability of non-DNA base-containing polynucleotides to stimulate TLR9 signaling. In contrast, it is known to those of ordinary skill in the art that CpG-containing polynucleotides stimulate TLR9 signaling, and that this signaling correlates with immune adjuvant activity.

### Example 3

### Immune Adjuvant Activity of Non-DNA Base-Containing Polynucleotides—Anti-HBsAg IgG Antibody Titers Following Immunization with Non-DNA Base-Containing Polynucleotides

The immune adjuvant activity of non-DNA base-containing polynucleotides was determined in a standard model (antibody response against hepatitis B surface antigen in mice). The immune response against this antigen is not to be regarded as only being specific for this antigen. On the contrary, the use of other antigens and other vaccine adjuvants known to those skilled in vaccine formulation and development is self-evident, and it is to be expected that this and other commonly known used approaches in vaccination can be used in the development of vaccines containing protein, peptide, carbohydrate and lipid antigens which are adjuvanted with non-DNA base-containing polynucleotides. The following examples illustrate how the use of non-DNA base-containing polynucleotides as vaccine adjuvants can elicit immune responses commonly considered as being protective in either prophylactic or therapeutic regimens.

Groups of 5 female BALB/c mice were immunized using a intramuscular (I.M.) route of administration on day 0, 21 and 31 with recombinant hepatitis B surface antigen (recombinant HBsAg; Cortex Biochemical, San Leandro, Calif., USA) and different combinations of aluminum hydroxide gel (Alum; SuperFos Biosector, Vedback, Denmark) and/or SEQ ID NOs: 1-3 (NebGGTGNeb, UUGTUU and IIGTII respectively). Complexation to alum was achieved by mixing the antigen and the non-DNA base-containing polynucleotide with alum prior to immunization. Sera were collected at day 41 and analyzed for the presence of anti-HBsAg IgG total, IgG1, IgG2a and IgG2b antibody by ELISA using HBsAg (1 μg/well, 96 well microtiter plates) and goat anti-mouse IgG, IgG1, IgG2a and IgG2b antibodies conjugated to HRP (Southern Biotechnologies, Birmingham, Ala.). Antibody titers were determined as end-point titers. The results of a typical immunization study are shown in Tables 2-5.

TABLE 2
Anti-HBsAg IgG (total) titers in BALB/c mice following
immunization with HBsAg in combination Alum and/or
non-DNA base-containing polynucleotides.
IgG total

| | | Anti-HBsAg titers* | |
|---|---|---|---|
| | | | Standard |
| Group | Immunization protocol | Mean | derivation |
| 1 | Control saline | 16 | 22 |
| 2 | HBsAg 1 μg | 80 630 | 98 421 |
| 3 | HBsAg 1 μg + Alum 10 μg | 157 649 | 107 948 |
| 4 | HBsAg 1 μg + SEQ ID NO: 1 10 μg | 109 647 | 57 962 |
| 5 | HBsAg 1 μg + SEQ ID NO: 1 10 μg + | 127 545 | 84 082 |
| | alum 10 μg | | |
| 6 | HBsAg 1 μg + SEQ ID NO: 2 10 μg | 69 812 | 24 792 |
| 7 | HBsAg 1 μg + SEQ ID NO: 2 10 μg + | 438 156 | 221 991 |
| | alum 10 μg | | |
| 8 | HBsAg 1 μg + SEQ ID NO: 3 10 μg | 147 941 | 152 424 |
| 9 | HBsAg 1 μg + SEQ ID NO: 3 10 μg + | 171 852 | 90 252 |
| | alum 10 μg | | |

*5 mice per group

TABLE 3
Anti-HBsAg IgG1 titers in BALB/c mice following
immunization with HBsAg in combination Alum and/or
non-DNA base-containing polynucleotides.
IgG1

| | | Anti-HBsAg titers* | |
|---|---|---|---|
| | | | Standard |
| Group | Immunization protocol | Mean | derivation |
| 1 | Control saline | 0 | 0 |
| 2 | HBsAg 1 μg | 52 929 | 60 268 |
| 3 | HBsAg 1 μg + Alum 10 μg | 100 395 | 66 606 |
| 4 | HBsAg 1 μg + SEQ ID NO: 1 10 μg | 88 491 | 53 085 |
| 5 | HBsAg 1 μg + SEQ ID NO: 1 10 μg + | 237 930 | 206 102 |
| | alum 10 μg | | |
| 6 | HBsAg 1 μg + SEQ ID NO: 2 10 μg | 60 086 | 16 198 |
| 7 | HBsAg 1 μg + SEQ ID NO: 2 10 μg + | 257 454 | 220 364 |
| | alum 10 μg | | |
| 8 | HBsAg 1 μg + SEQ ID NO: 3 10 μg | 112 141 | 107 727 |
| 9 | HBsAg 1 μg + SEQ ID NO: 3 10 μg + | 185 656 | 172 171 |
| | alum 10 μg | | |

*5 mice per group

TABLE 4
Anti-HBsAg IgG2a titers in BALB/c mice following
immunization with HBsAg in combination Alum and/or
non-DNA base-containing polynucleotides.
IgG2a

| | | Anti-HBsAg titers* | |
|---|---|---|---|
| | | | Standard |
| Group | Immunization protocol | Mean | derivation |
| 1 | Control saline | 4 | 0 |
| 2 | HBsAg 1 μg | 349 | 498 |
| 3 | HBsAg 1 μg + Alum 10 μg | 3 276 | 4 169 |
| 4 | HBsAg 1 μg + SEQ ID NO: 1 10 μg | 1 850 | 1 504 |
| 5 | HBsAg 1 μg + SEQ ID NO: 1 10 μg + | 26 268 | 55 568 |
| | alum 10 μg | | |
| 6 | HBsAg 1 μg + SEQ ID NO: 2 10 μg | 3 595 | 5 394 |
| 7 | HBsAg 1 μg + SEQ ID NO: 2 10 μg + | 10 722 | 12 038 |
| | alum 10 μg | | |
| 8 | HBsAg 1 μg + SEQ ID NO: 3 10 μg | 5 217 | 7 557 |
| 9 | HBsAg 1 μg + SEQ ID NO: 3 10 μg + | 12 533 | 5 280 |
| | alum 10 μg | | |

*5 mice per group

TABLE 5
Anti-HBsAg IgG2b titers in BALB/c mice following
immunization with HBsAg in combination Alum and/or
non-DNA base-containing polynucleotides.
IgG2b

| | | Anti-HBsAg titers* | |
|---|---|---|---|
| | | | Standard |
| Group | Immunization protocol | Mean | derivation |
| 1 | Control saline | 0 | 0 |
| 2 | HBsAg 1 μg | 862 | 1 422 |
| 3 | HBsAg 1 μg + Alum 10 μg | 3 217 | 3 565 |
| 4 | HBsAg 1 μg + SEQ ID NO: 1 10 μg | 3 430 | 3 586 |
| 5 | HBsAg 1 μg + SEQ ID NO: 1 10 μg + | 46 602 | 97 608 |
| | alum 10 μg | | |
| 6 | HBsAg 1 μg + SEQ ID NO: 2 10 μg | 2 864 | 2 735 |
| 7 | HBsAg 1 μg + SEQ ID NO: 2 10 μg + | 13 540 | 11 945 |
| | alum 10 μg | | |
| 8 | HBsAg 1 μg + SEQ ID NO: 3 10 μg | 3 265 | 2 572 |
| 9 | HBsAg 1 μg + SEQ ID NO: 3 10 μg + | 9 251 | 3 970 |
| | alum 10 μg | | |

*5 mice per group

The results demonstrate immunoadjuvant activity for non-DNA base-containing polynucleotides. In this example, SEQ ID NOs: 1-3 demonstrated significant immunoadjuvant activity for three non-DNA base-containing polynucleotides, as determined by the anti-antigen IgG antibody titer when complexed to alum, and that the antibody titers achieved are higher than those seen for antigen alone or antigen complexed to the standard adjuvant alum. Of greater significance is the demonstration that although the use of alum enhances the immune adjuvant activity of the non-DNA base-containing polynucleotides, the non-DNA base-containing polynucleotides can be used without a particulate carrier in combination with a soluble antigen as a means of eliciting higher levels of antigen-specific antibodies. Of greater significance is the observation that the use of non-DNA base-containing polynucleotides as adjuvants, either alone or in combination with an alum carrier, results in an increase in the level of IgG antibodies of the IgG2a and IgG2b isotype. These antibodies are recognized by those skilled in the art as being particularly advantageous when eliciting an immune response against viral antigens.

### Example 4

### Immune Adjuvant Activity of Non-DNA Base-Containing Polynucleotides—Anti-H1n1virion IgG Antibody Titers Following Immunization with Non-DNA Base-Containing Polynucleotides

The immune adjuvant activity of non-DNA base-containing polynucleotides was determined in a standard model (antibody response against H1N1 virions in mice). The immune response against this antigen is not to be regarded as only being specific for this virion. On the contrary, the use of other virions and other vaccine adjuvants known to those skilled in vaccine formulation and development is self-evident, and it is to be expected that this and other commonly known used approaches in vaccination can be used in the development of vaccines containing protein, peptide, carbohydrate and lipid antigens which are adjuvanted with non-DNA base-containing polynucleotides. The following example illustrates how the use of non-DNA base-containing polynucleotides as vaccine adjuvants can elicit immune responses commonly considered as being protective in either prophylactic or therapeutic regimens.

Groups of 5 female C57BL/6 mice were immunized using a intramuscular (I.M.) route of administration on day 0, 21 and 31 with H1N1 virions (Genway Biotech, San Diego, Calif., USA, Influenza type A Beijing H1N1) and different combinations of aluminum hydroxide gel (Alum; SuperFos Biosector, Vedback, Denmark) and/or SEQ ID NOs: 1 to 9. Complexation to alum was achieved by mixing the H1N1 virion and the non-DNA base-containing polynucleotide with alum prior to immunization. Sera were collected at day 41 and analyzed for the presence of anti-H1N1 virion IgG1 and IgG2b antibodies by standard ELISA using H1N1 virions (1 μg/well, 96 well microtiter plates) and goat anti-mouse IgG1 and IgG2b antibodies conjugated to HRP (Southern Biotechnologies, Birmingham, Ala.). Antibody titers were determined as end-point titers. The results of a typical immunization study are shown in Tables 6-7.

TABLE 6
Anti-H1N1 virion IgG1 antibody titers in C57/BL-6 female mice
following immunization with H1N1 virions in combination with
Alum and/or non-DNA base-containing polynucleotides.
IgG1

| | | Anti-H1N1 titers* | |
|---|---|---|---|
| | | | Standard |
| Group | Immunization protocol | Mean | derivation |
| 1 | Control saline | 44 | 98 |
| 2 | H1N1 1 μg | 9554 | 7156 |
| 3 | H1N1 1 μg + Alum 10 μg | 47422 | 24890 |
| 4 | H1N1 1 μg + SEQ ID NO: 1 10 μg | 2270 | 2215 |
| 5 | H1N1 1 μg + Alum 10 μg + | 69528 | 38017 |
| | SEQ ID NO: 1 10 μg | | |
| 6 | H1N1 1 μg + SEQ ID NO: 2 10 μg | 3863 | 1984 |
| 7 | H1N1 1 μg + Alum 10 μg + | 26359 | 14472 |
| | SEQ ID NO: 2 10 μg | | |
| 8 | H1N1 1 μg + SEQ ID NO: 3 10 μg | 3508 | 2069 |
| 9 | H1N1 1 μg + Alum 10 μg + | 22238 | 9392 |
| | SEQ ID NO: 3 10 μg | | |
| 10 | H1N1 1 μg + SEQ ID NO: 4 10 μg | 8435 | 11344 |
| 11 | H1N1 1 μg + Alum 10 μg + | 53768 | 28138 |
| | SEQ ID NO: 4 10 μg | | |
| 12 | H1N1 1 μg + SEQ ID NO: 5 10 μg | 6551 | 4870 |
| 13 | H1N1 1 μg + Alum 10 μg + | 30316 | 24590 |
| | SEQ ID NO: 5 10 μg | | |
| 14 | H1N1 1 μg + SEQ ID NO: 6 10 μg | 15566 | 22080 |
| 15 | H1N1 1 μg + Alum 10 μg + | 21634 | 17778 |
| | SEQ ID NO: 6 10 μg | | |
| 16 | H1N1 1 μg + SEQ ID NO: 7 10 μg | 15567 | 32622 |
| 17 | H1N1 1 μg + Alum 10 μg + | 29788 | 28747 |
| | SEQ ID NO: 7 10 μg | | |
| 18 | H1N1 1 μg + SEQ ID NO: 8 10 μg | 2477 | 3348 |
| 19 | H1N1 1 μg + Alum 10 μg + | 28214 | 18376 |
| | SEQ ID NO: 8 10 μg | | |
| 20 | H1N1 1 μg + SEQ ID NO: 9 10 μg | 2249 | 1619 |
| 21 | H1N1 1 μg + Alum 10 μg + | 18956 | 13527 |
| | SEQ ID NO: 9 10 μg | | |

*5 mice per group

TABLE 7
Anti-H1N1 virion IgG2b antibody titers in C57/BL-6 female mice
following immunization with H1N1 virions in combination with
Alum and/or non-DNA base-containing polynucleotides.
IgG2b

| | | Anti-H1N1 titers* | |
|---|---|---|---|
| | | | Standard |
| Group | Immunization protocol | Mean | derivation |
| 1 | Control saline | 0 | 0 |
| 2 | H1N1 1 μg | 8179 | 2411 |
| 3 | H1N1 1 μg + Alum 10 μg | 12152 | 8921 |
| 4 | H1N1 1 μg + SEQ ID NO: 1 10 μg | 6521 | 3000 |
| 5 | H1N1 1 μg + Alum 10 μg + | 12429 | 7764 |
| | SEQ ID NO: 1 10 μg | | |
| 9 | H1N1 1 μg + SEQ ID NO: 2 10 μg | 22994 | 18153 |
| 7 | H1N1 1 μg + Alum 10 μg + | 25112 | 18866 |
| | SEQ ID NO: 2 10 μg | | |
| 8 | H1N1 1 μg + SEQ ID NO: 3 10 μg | 12456 | 8189 |
| 9 | H1N1 1 μg + Alum 10 μg + | 18018 | 5225 |
| | SEQ ID NO: 3 10 μg | | |
| 10 | H1N1 1 μg + SEQ ID NO: 4 10 μg | 9420 | 9191 |
| 11 | H1N1 1 μg + Alum 10 μg + | 31431 | 30917 |
| | SEQ ID NO: 4 10 μg | | |
| 12 | H1N1 1 μg + SEQ ID NO: 5 10 μg | 19056 | 31508 |
| 13 | H1N1 1 μg + Alum 10 μg + | 19574 | 24662 |
| | SEQ ID NO: 5 10 μg | | |
| 14 | H1N1 1 μg + SEQ ID NO: 6 10 μg | 15967 | 11635 |
| 15 | H1N1 1 μg + Alum 10 μg + | 10552 | 4563 |
| | SEQ ID NO: 6 10 μg | | |
| 16 | H1N1 1 μg + SEQ ID NO: 7 10 μg | 14668 | 15155 |
| 17 | H1N1 1 μg + Alum 10 μg + | 25382 | 27791 |
| | SEQ ID NO: 7 10 μg | | |
| 18 | H1N1 1 μg + SEQ ID NO: 8 10 μg | 5776 | 2810 |
| 19 | H1N1 1 μg + Alum 10 μg + | 10431 | 2921 |
| | SEQ ID NO: 8 10 μg | | |
| 20 | H1N1 1 μg + SEQ ID NO: 9 10 μg | 3634 | 1990 |
| 21 | H1N1 1 μg + Alum 10 μg + | 8683 | 6266 |
| | SEQ ID NO: 9 10 μg | | |

*5 mice per group

The results shown in Tables 6 demonstrate that non-DNA base-containing polynucleotide SEQ ID NOs: 1, 2, 3, 5 and 9 have the capacity to reduce IgG1 antibody titers when administered with the H1N1 virions alone. The non-DNA base-containing polynucleotide SEQ ID NOs: 6 and 7 stimulated anti-IgG1 antibody levels when administered with the H1N1 virions alone. SEQ ID NO: 8 had no effect on anti-IgG1 antibody titers when administered with the H1N1 virions alone. When H1N1 virions were complexed to alum and the non-DNA base-containing polynucleotides only SEQ ID NOs: 2 and 4 gave IgG1 antibody titers that were elevated when compared to H1N1 complexed to alum alone.

These data demonstrate that it is possible to attenuate or enhance IgG1 responses against H1N1 by using the appropriate non-DNA base-containing polynucleotide and formulation (soluble formulation in saline or complexed to the carrier alum). The results shown in Table 7 demonstrate that non-DNA base-containing polynucleotide SEQ ID NOs: 1, 8 and 9 have the capacity to reduce IgG2b antibody titers when administered with the H1N1 virions alone. The non-DNA base-containing polynucleotide SEQ ID NOs: 2, 3, 5, 6 and 7 stimulated anti-IgG1 antibody levels when administered with the H1N1 virions alone. SEQ ID NO: 4 had no effect on anti-IgG2b antibody titers when administered with the H1N1 virions alone. When H1N1 virions were complexed to alum and the non-DNA base-containing polynucleotides SEQ ID NOs: 1, 2, 3, 4, 5 and 7 gave antibody titers that were elevated when compared to H1N1 complexed to alum alone. SEQ ID NOs: 6, 8 and 9 had no effect on IgG2b anti-H1N1 antibody levels when complexed to alum. These data demonstrate that it is possible to attenuate or enhance IgG2b responses against H1N1virions by using the appropriate non-DNA base-containing polynucleotide and formulation (soluble formulation in saline or complexed to the carrier alum).

### Example 5

### Immune Adjuvant Activity of Non-DNA Base-Containing Polynucleotides—Anti-H1N1 virion IgG Antibody Titers Following Immunization with Non-DNA Base-Containing Polynucleotides—Antigen Sparing

The ability of non-DNA base-containing polynucleotides to function as antigen-sparing adjuvants was evaluated using the H1N1 virion model system in mice. An abbreviated immunization schedule and reduced virion challenge dose were deliberately selected to represent the vaccination constraints that would be encountered during a pandemic influenza outbreak. Groups of 5 female C57BL/6 mice were immunized using the intramuscular (I.M.) route of administration on day 0 and 14 with H1N1 virions at immunization doses of 0.1 and 0.01 μg/injection (Genway Biotech, San Diego, Calif., USA, Influenza A Beijing H1N1) and different combinations of aluminum hydroxide gel (Alum; SuperFos Biosector, Vedback, Denmark) and/or SEQ ID NOs: 1, 3, 4, 5, 6 and 7. Complexation to alum was achieved by mixing the H1N1 virion and the non-DNA base-containing polynucleotide with alum prior to immunization. Sera were collected at day 22 and analyzed for the presence of anti-H1N1 virion IgG2b antibodies by standard ELISA using H1N1 virions (1 μg/well, 96 well microtiter plates) and goat anti-mouse IgG1 and IgG2b antibodies conjugated to HRP (Southern Biotechnologies, Birmingham, Ala.). Antibody titers were determined as end-point titers. The results of a typical immunization study are shown in Tables 8-11.

TABLE 8
Anti-H1N1 virion IgG1 antibody titers in C57/BL-6 female mice
following immunization with 0.1 μg H1N1 virions in combination
with Alum and/or non-DNA base-containing polynucleotides.
IgG1

| | | Anti-H1N1 titers* | |
|---|---|---|---|
| | | | Standard |
| Group | Immunization protocol | Mean | derivation |
| 1 | Control saline | 4 | 80 |
| 2 | H1N1 0.1 μg | 458 | 497 |
| 3 | H1N1 0.1 μg + Alum 10 μg | 1538 | 2320 |
| 4 | H1N1 0.1 μg + SEQ ID NO: 1 10 μg | 594 | 834 |
| 5 | H1N1 0.1 μg + Alum 10 μg + | 11054 | 6882 |
| | SEQ ID NO: 1 10 μg | | |
| 6 | H1N1 0.1 μg + SEQ ID NO: 3 10 μg | 168 | 234 |
| 7 | H1N1 0.1 μg + Alum 10 μg + | 2907 | 3422 |
| | SEQ ID NO: 3 10 μg | | |
| 8 | H1N1 0.1 μg + SEQ ID NO: 4 10 μg | 781 | 567 |
| 9 | H1N1 0.1 μg + Alum 10 μg + | 28908 | 27948 |
| | SEQ ID NO: 4 10 μg | | |
| 10 | H1N1 0.1 μg + SEQ ID NO: 5 10 μg | 65 | 110 |
| 11 | H1N1 0.1 μg + Alum 10 μg + | 6519 | 8183 |
| | SEQ ID NO: 5 10 μg | | |
| 12 | H1N1 0.1 μg + SEQ ID NO: 6 10 μg | 866 | 820 |
| 13 | H1N1 0.1 μg + Alum 10 μg + | 9066 | 5870 |
| | SEQ ID NO: 6 10 μg | | |
| 14 | H1N1 0.1 μg + SEQ ID NO: 7 10 μg | 29 | 60 |
| 15 | H1N1 0.1 μg + Alum 10 μg + | 6327 | 3945 |
| | SEQ ID NO: 7 10 μg | | |

*5 mice per group

TABLE 9
Anti-H1N1 virion IgG2b antibody titers in C57/BL-6 female mice
following immunization with 0.1 μg H1N1 virions in combination
with Alum and/or non-DNA base-containing polynucleotides.
IgG2b

| | | Anti-H1N1 titers* | |
|---|---|---|---|
| | | | Standard |
| Group | Immunization protocol | Mean | derivation |
| 1 | Control saline | 26 | 59 |
| 2 | H1N1 0.1 μg | 206 | 267 |
| 3 | H1N1 0.1 μg + Alum 10 μg | 700 | 1097 |
| 4 | H1N1 0.1 μg + SEQ ID NO: 1 10 μg | 659 | 506 |
| 5 | H1N1 0.1 μg + Alum 10 μg + | 659 | 506 |
| | SEQ ID NO: 1 10 μg | | |
| 6 | H1N1 0.1 μg + SEQ ID NO: 3 10 μg | 65 | 126 |
| 7 | H1N1 0.1 μg + Alum 10 μg + | 971 | 1282 |
| | SEQ ID NO: 3 10 μg | | |
| 8 | H1N1 0.1 μg + SEQ ID NO: 4 10 μg | 355 | 267 |
| 9 | H1N1 0.1 μg + Alum 10 μg + | 2598 | 1825 |
| | SEQ ID NO: 4 10 μg | | |
| 10 | H1N1 0.1 μg + SEQ ID NO: 5 10 μg | 0 | 0 |
| 11 | H1N1 0.1 μg + Alum 10 μg + | 2156 | 3222 |
| | SEQ ID NO: 5 10 μg | | |
| 12 | H1N1 0.1 μg + SEQ ID NO: 6 10 μg | 715 | 776 |
| 13 | H1N1 0.1 μg + Alum 10 μg + | 3893 | 3112 |
| | SEQ ID NO: 6 10 μg | | |
| 14 | H1N1 0.1 μg + SEQ ID NO: 7 10 μg | 10 | 11 |
| 15 | H1N1 0.1 μg + Alum 10 μg + | 951 | 1056 |
| | SEQ ID NO: 7 10 μg | | |

*5 mice per group

TABLE 10
Anti-H1N1 virion IgG1 antibody titers in C57/BL-6 female mice
following immunization with 0.01 μg H1N1 virions in combination
with Alum and/or non-DNA base-containing polynucleotides.
IgG1

| | | Anti-H1N1 titers* | |
|---|---|---|---|
| | | | Standard |
| Group | Immunization protocol | Mean | derivation |
| 1 | Control saline | 04 | 8 |
| 2 | H1N1 0.01 μg | 86 | 183 |
| 3 | H1N1 0.01 μg + Alum 10 μg | 7 | 15 |
| 4 | H1N1 0.01 μg + SEQ ID NO: 1 10 μg | 20 | 44 |
| 5 | H1N1 0.01 μg + Alum 10 μg + | 451 | 694 |
| | SEQ ID NO: 1 10 μg | | |
| 6 | H1N1 0.01 μg + SEQ ID NO: 3 10 μg | 28 | 38 |
| 7 | H1N1 0.01 μg + Alum 10 μg + | 203 | 242 |
| | SEQ ID NO: 3 10 μg | | |
| 8 | H1N1 0.01 μg + SEQ ID NO: 4 10 μg | 337 | 542 |
| 9 | H1N1 0.01 μg + Alum 10 μg + | 0 | 0 |
| | SEQ ID NO: 4 10 μg | | |
| 10 | H1N1 0.01 μg + SEQ ID NO: 5 10 μg | 78 | 174 |
| 11 | H1N1 0.01 μg + Alum 10 μg + | 580 | 534 |
| | SEQ ID NO: 5 10 μg | | |
| 12 | H1N1 0.01 μg + SEQ ID NO: 6 10 μg | 31 | 67 |
| 13 | H1N1 0.01 μg + Alum 10 μg + | 1628 | 2067 |
| | SEQ ID NO: 6 10 μg | | |
| 14 | H1N1 0.01 μg + SEQ ID NO: 7 10 μg | 0 | 0 |
| 15 | H1N1 0.01 μg + Alum 10 μg + | 247 | 457 |
| | SEQ ID NO: 7 10 μg | | |

*5 mice per group

TABLE 11
Anti-H1N1 virion IgG2b antibody titers in C57/BL-6 female mice
following immunization with 0.01 μg H1N1 virions in combination
with Alum and/or non-DNA base-containing polynucleotides.
IgG2b

| | | Anti-H1N1 titers* | |
|---|---|---|---|
| | | | Standard |
| Group | Immunization protocol | Mean | derivation |
| 1 | Control saline | 026 | 590 |
| 2 | H1N1 0.01 μg | 13 | 29 |
| 3 | H1N1 0.01 μg + Alum 10 μg | 3 | 6 |
| 4 | H1N1 0.01 μg + SEQ ID NO: 1 10 μg | 3 | 8 |
| 5 | H1N1 0.01 μg + Alum 10 μg + | 150 | 208 |
| | SEQ ID NO: 1 10 μg | | |
| 6 | H1N1 0.01 μg + SEQ ID NO: 3 10 μg | 80 | 125 |
| 7 | H1N1 0.01 μg + Alum 10 μg + | 302 | 575 |
| | SEQ ID NO: 3 10 μg | | |
| 8 | H1N1 0.01 μg + SEQ ID NO: 4 10 μg | 9 | 19 |
| 9 | H1N1 0.01 μg + Alum 10 μg + | 48 | 52 |
| | SEQ ID NO: 4 10 μg | | |
| 10 | H1N1 0.01 μg + SEQ ID NO: 5 10 μg | 1 | 2 |
| 11 | H1N1 0.01 μg + Alum 10 μg + | 34 | 75 |
| | SEQ ID NO: 5 10 μg | | |
| 12 | H1N1 0.01 μg + SEQ ID NO: 6 10 μg | 0 | 0 |
| 13 | H1N1 0.01 μg + Alum 10 μg + | 1149 | 1593 |
| | SEQ ID NO: 6 10 μg | | |
| 14 | H1N1 0.01 μg + SEQ ID NO: 7 10 μg | 3 | 6 |
| 15 | H1N1 0.01 μg + Alum 10 μg + | 1 | 2 |
| | SEQ ID NO: 7 10 μg | | |

*5 mice per group

The results shown in Tables 8-11 demonstrate that by adjuvanting H1N1 virions at low virion challenge doses of 0.1 or 0.01 μg/injection with non-DNA base-containing polynucleotides, and using an abbreviated immunization protocol, it is still possible to elicit anti-H1N1 antibodies of the IgG1 and IgG2b class under conditions where H1N1, either alone or complexed to alum, showed marginal to no immunogenicity. Optimum antibody responses were obtained using alum as the carrier for the virions and for the non-DNA base-containing polynucleotides. Non-DNA base-containing polynucleotides can therefore be used as antigen-sparing adjuvants even when used with less than optimal immunization schedules.

### Example 6

### Immune Adjuvant Activity of Non-DNA Base-Containing Polynucleotides—Anti-Human Serum Albumin IgG Antibody Titers Following Immunization with Non-DNA Base-Containing Polynucleotides

The immune adjuvant activity of non-DNA base-containing polynucleotides using a protein antigen (as a model representative of sub-unit antigenic protein vaccines) was investigated using human serum albumin in mice. The study was intended to determine the effect of immunizing with soluble antigen and soluble peptide in the absence of a vaccine carrier adjuvant such as alum. Groups of 5 female C57BL/6 mice (Charles River Laboratories Inc., St. Constant, Québec, Canada) were immunized using the intramuscular (I.M.) route of administration on day 0 and 21 with human serum albumin (HSA, Sigma-Aldrich Canada, Oakville, Ontario, Canada #A9731, 28.5 mg/mL) at an immunization dose of 1 μg/injection without or in combination with SEQ ID NOs: 1, 2, 3, 4, 5, 6, 7, 8 and 9. The polynucleotides were simply mixed with HSA in saline prior to immunization, no particulate carrier system being used. Sera were collected at day 29-30 and analyzed for the presence of anti-HAS IgG antibodies by standard ELISA using HSA (1 μg/well, 96 well microtiter plates) and goat anti-mouse IgG antibodies conjugated to HRP (Southern Biotechnologies, Birmingham, Ala.). IgG anti-HSA antibody titers were determined as end-point titers. The results of a typical immunization study are shown in Tables 12.

TABLE 12
Anti-HSA IgG antibody titers in C57/BL-6 female
mice following immunization with 1 μg HSA
and/or non-DNA base-containing polynucleotides.
IgG total

| | | Anti-H1N1 titers* | |
|---|---|---|---|
| | | | Standard |
| Group | Immunization protocol | Mean | derivation |
| 1 | Control saline | 0 | 0 |
| 2 | HSA 1 μg | 1888 | 4004 |
| 3 | HSA 1 μg + SEQ ID NO: 1 10 μg | 88 | 164 |
| 4 | HSA 1 μg + SEQ ID NO: 2 10 μg | 0 | 0 |
| 5 | HSA 1 μg + SEQ ID NO: 3 10 μg | 364 | 586 |
| 6 | HSA 1 μg + SEQ ID NO: 4 10 μg | 5662 | 9129 |
| 7 | HSA 1 μg + SEQ ID NO: 5 10 μg | 14516 | 25511 |
| 8 | HSA 1 μg + SEQ ID NO: 6 10 μg | 4003 | 5651 |
| 9 | HSA 1 μg + SEQ ID NO: 7 10 μg | 168 | 346 |
| 10 | HSA 1 μg + SEQ ID NO: 8 10 μg | 2 | 5 |
| 11 | HSA 1 μg + SEQ ID NO: 9 10 μg | 0 | 0 |

*5 mice per group

The results of this study demonstrate that mixing HSA with the polynucleotides in saline resulted in 2 types of adjuvant effect. Firstly, SEQ ID NOs: 1, 2, 3, 7, 8 and 9 resulted in a decrease in IgG antibody titers when compared to HSA alone. Secondly, SEQ ID NOs: 4, 5 and 6 resulted in an increase in IgG antibody titers when compared to HSA alone. It is clear from these data that non-DNA base-containing polynucleotides have the capacity to act as immune modulators with respect to immune adjuvant activity (that is, inhibition or stimulation of antibody responses) when administered as a composition with an antigen in their soluble form as 5′OH, 3′OH unprotected, phosphodiester non-DNA base-containing polynucleotides.

### Example 7

### Induction of Cytokines from Human PBMC Cells by Non-DNA Base-Containing Polynucleotides

A number of immune adjuvants are known to be capable of eliciting cytokines from human PBMC cells. Examples of such adjuvants are alum, lipopolysaccharide, monophosphoryl lipid A, CpG-containing polynucleotides and non-CpG-containing ODN's, as well as mycobacterial cell wall-DNA complex (MCC). The ability of non-DNA base-containing polynucleotides, specifically, SEQ ID NOs: 1-3 (1-100 μg/mL) to induce cytokines from human PBMC obtained from 3 healthy adult individuals was determined. PBMC were isolated from whole blood using standard Ficoll-Paque (GE Healthcare Life Sciences, Baie d'Urfé, QC, Canada) gradient centrifugation, and placed in tissue culture plates at a concentration of 10⁶ cells/mL (total volume: 1 mL in RPMI 1640 medium containing 10% heat-inactivated FBS, both from Wisent, St-Bruno, QC, Canada) and 50 μg/mL gentamycin sulfate (Sigma-Aldrich Canada, Oakville, ON, Canada). SEQ ID NOs: 1, 2 or 3 were added to give a final concentration of 100 μg/mL, and the cells were cultivated for 48 h. Cytokines (IL-1β, IL-2, IL-6, IL-10 and IL-12p40) in the supernatant at the end of incubation were determined by ELISA using a cytokine capture assay process (Biosource, Camarillo, Calif.). SEQ ID NO: 32 CpG 2006 ODN (1-100 μg/mL, 5′-TCGTCGTTTTGTCGTTTTGTCGTT-3′, A-class CpG), MCC (1-100 μg/mL) and LPS (10 μg/mL) were used as positive controls. The results of the cytokine capture assay are shown in Table 13.

TABLE 13
Induction of cytokine synthesis in human PBMC
by non-DNA base-containing polynucleotides

| | Number of individuals responding¹ | | | | |
|---|---|---|---|---|---|
| Treatment | IL-1β | IL-2 | IL-6 | IL-10 | IL-12p40 |
| SEQ ID NO: 1 | 3/3 | 0/3 | 3/3 | 0/3 | 3/3 |
| SEQ ID NO: 2 | 0/3 | 0/3 | 1/3 | 0/3 | 3/3 |
| SEQ ID NO: 3 | 0/3 | 0/3 | 3/3 | 0/3 | 0/3 |
| SEQ ID NO: 32 CpG 2006 | 0/3 | 0/3 | 3/3 | 2/3 | 1/3 |
| MCC | 3/3 | 0/3 | 3/3 | 3/3 | 3/3 |
| LPS | 3/3 | 0/3 | 3/3 | 3/3 | 3/3 |

¹The results are expressed as the number of individuals responding with an increase in cytokine levels two-fold that of the background control treatment.

The results demonstrate that non-DNA base-containing polynucleotides have a cytokine synthesis stimulating profile that is distinct to that of CpG 2006, MCC or LPS. The levels of cytokines induced by the non-DNA base-containing polynucleotides of the present invention were also significantly lower than those induced by MCC or LPS (10-fold less), and whilst not wishing to be held to the following, such data strongly indicates that the ability to induce cytokines is not directly related to the immune adjuvant activity of non-DNA base-containing polynucleotides.

### Example 8

### Induction of Cytokines from Human PBMC, Comparison with Phosphorothioate CpG-Containing Polynucleotides

In a separate study, the ability of non-DNA base-containing polynucleotides (phosphodiester) to induce cytokine synthesis in human PBMC populations was determined and compared with that of a number of CpG-containing phosphorothioate polynucleotides. LPS and MCC were included as positive controls as described in Example 7. PBMC were isolated from whole blood using standard Ficoll-Paque Plus (GE Healthcare Life Sciences, Baie d'Urfé, QC, Canada) gradient centrifugation, and placed in tissue culture plates at a concentration of 1×10⁶ cells/mL (total volume: 1 mL in RPMI 1640 medium containing 10% heat-inactivated FBS (both from Wisent, St-Bruno, QC, Canada) and 50 μg/mL gentamycin sulfate (Sigma-Aldrich Canada, Oakville, ON, Canada). Non-DNA base-containing polynucleotide SEQ ID NOs: 1, 2, 3, 4, 5, 6, 7, 8 and 9 were added to separate wells to give a final concentration of 10 μg/mL, LPS was added to give a final concentration of 10 μg/mL, and the non-CpG 4010, CpG 2336, CpG 2429 and CpG 7909 polynucleotides, SEQ ID NOs: 31, 33, 35 and 34, respectively, were added to give a final concentration of 10 μg/mL.

The cells were then cultivated for 48 h. Cytokines (interleukins IL-6 and IL-10, chemokines MCP-1 and RANTES) in the culture supernatant were measured using a Milliplex™ MAP kit (Millipore Corporation, Billerica, Mass., USA) on a Bio-Plex 200 system (Bio-Rad Laboratories, Hercules, Calif., USA). MCP-1, also known as monocyte chemotactic protein, is an essential chemokine required for effective monocyte and myeloid dendritic cell activation and trafficking across endothelial and epithelial barriers (see for example Henkel et al, 2004 Ann. Neurol. 55:221-235), and RANTES (Regulated on Activation, Normal T cell Expressed and Secreted, now more commonly known as CCL5) is produced by memory phenotype T cells and is chemotactic for and activates T-cells (see for example Swanson et al, 2002 Immunity 17:605-615). The results of the study are shown in Table 14.

TABLE 14
Induction of cytokine synthesis in human PBMC,
comparison with CpG-containing polynucleotides

| | Cytokine, pg/mL | | | |
|---|---|---|---|---|
| Treatment | IL-6 | IL-10 | MCP-1 | RANTES (CCL5) |
| Control | 0 | 0 | 32 | 70 |
| LPS | 6337 | 2 | 1063 | 121 |
| MCC | 738 | 14 | 6556 | 178 |
| SEQ ID NO: 1 | 26 | 0 | 160 | 93 |
| SEQ ID NO: 2 | 6 | 0 | 196 | 66 |
| SEQ ID NO: 3 | 1 | 0 | 57 | 72 |
| SEQ ID NO: 4 | 1960 | 28 | 7000 | 211 |
| SEQ ID NO: 5 | 845 | 13 | 3600 | 202 |
| SEQ ID NO: 6 | 0 | 0 | 48 | 137 |
| SEQ ID NO: 7 | 1 | 0 | 79 | 71 |
| SEQ ID NO: 8 | 0 | 0 | 78 | 78 |
| SEQ ID NO: 9 | 133 | 1 | 914 | 117 |
| SEQ ID NO: 31 | 43 | 0 | 343 | 275 |
| SEQ ID NO: 33 | 9 | 0 | 573 | 105 |
| SEQ ID NO: 34 | 41 | 2 | 1270 | 187 |
| SEQ ID NO: 35 | 42 | 0 | 906 | 136 |

The results of this study show that in comparison to the immune stimulants LPS and MCC, only the SEQ ID NO: 4 and 5 show both strong interleukin (IL-6) and chemokine-inducing activity (MCP-1, RANTES) towards human PBMC. SEQ ID NO: 9 only showed strong MCP-1 inducing activity. The SEQ ID NOs: 1, 2, 3, 6, 7 and 8 showed marginal or no interleukin and marginal or no chemokine inducing activity. The CpG polynucleotides (SEQ ID NOs: 33, 34, 35) did not induce cytokines, but all had strong MCP-1 inducing activity. It should also be noted that the Non-CpG 4010 polynucleotide SEQ ID NO: 31 also had chemokine-inducing activity. It is clear from these data that SEQ ID NOs: 1 through 9 have interleukin and chemokine inducing profiles that are distinct to those of phosphorothioate backbone CpG polynucleotides, and that the ability to induce interleukins and chemokines does not correlate with the ability to act as an immune adjuvant as demonstrated in the preceding examples.

### Example 9

### Co-Adjuvantation of Vaccines Containing Alum with Non-DNA Base-Containing Polynucleotides

The following example illustrates the use of the polynucleotide sequences of the present invention in enhancing the immune response to conventional vaccines containing aluminum salts (alum) used as antigen carrier and adjuvant. Alum is regarded as a safe adjuvant for population-based immunization procedures. A large proportion of number of commercially available vaccines are adjuvanted with aluminum salts (such as but not limited to aluminum hydroxide, aluminum phosphate or aluminum sulfate). These vaccines are further adjuvanted using non-DNA base-containing polynucleotides of the present invention such that a stimulation of antibody production and protective activity is achieved. The dose of non-DNA base-containing polynucleotides used (within but not limited to the range 1-100 μg/vaccine dose) is added to the vaccine either during the manufacturing process during the aluminum complexation stage or immediately pre-immunization, and immunization of individuals is carried out according to established vaccination procedures using established vaccine doses and routes of administration (conventionally intramuscular into the deltoid muscle). Immunization with aluminum-adjuvanted vaccines containing non-DNA base-containing polynucleotides results in a superior immune response when compared to vaccination with aluminum-adjuvanted vaccines, as determined by enhanced protective antibody titers and resistance to infectious exposure or elimination of established disease. One of skill in the art will recognize that a) benefit from the enhanced immune response is also achieved when the number of immunizations is not optimal (for example 2 instead of 3 immunizations) or when the dose of antigen(s) administered is less than optimal, as for example during pandemic immunization, and b) that antigen sparing in vaccines adjuvanted with alum will result from co-adjuvantation with non-DNA base-containing polynucleotides. Examples of vaccines that benefit from the addition of non-DNA base-containing polynucleotides are (but not limited to) DTaP (diphtheria-tetanus-pertussis) vaccines, Hepatitis A vaccines, hepatitis A+B, Hib (haemophilus influenza B) vaccines, DTaP-IPV (inactivated polio virus)-hepatitis B, pneumococcal conjugate, DT (diphtheria-tetanus) vaccines, Hib-hepatitis B vaccines, HPV (human papilloma virus) vaccines, H1N1 vaccines, and rabies vaccines.

### Example 10

### Co-Adjuvantation of Vaccines Containing Oil-Based Adjuvants with Non-DNA Base-Containing Polynucleotides

The following example illustrates the use of the polynucleotide sequences of the present invention in enhancing the immune response to conventional vaccines containing oil or oil-based emulsions as adjuvant. A number of commercially available vaccines, especially those used in veterinary practice, are adjuvanted with oil or oil-based emulsions. These oils comprise but are not limited to mineral oil, montanides or squalene. These vaccines are further adjuvanted using non-DNA base-containing polynucleotides of the present invention such that a stimulation of antibody production and protective activity is achieved. The dose of non-DNA base-containing polynucleotides used (within but not limited to the range 1-100 μg/vaccine dose) is added to the vaccine either during the manufacturing process during the formulation stage or immediately pre-immunization, and immunization of individuals is carried according to established vaccination procedures using established vaccine doses and routes of administration (conventionally but not limited to intramuscular injection). Immunization of individuals with oil or oil-based emulsion adjuvanted vaccines containing non-DNA base-containing polynucleotides results in a superior immune response when compared to vaccination with oil or oil-based emulsion adjuvanted vaccines alone, as determined by enhanced protective antibody titers, resistance to infectious organism exposure or elimination of established disease. One of skill in the art will recognize that a) benefit from the enhanced immune response is also achieved when the number of immunizations is not optimal (for example 1 or 2 immunizations instead of 3 or more immunizations) or when the dose of antigen(s) administered is less than optimal, as for example during pandemic immunization, and b) that antigen sparing in vaccines oil or oil-based emulsion adjuvanted vaccines will result from co-adjuvantation with non-DNA base-containing polynucleotides. Examples of vaccines that are adjuvanted with oil or oil-based emulsions that benefit from the addition of non-DNA base-containing polynucleotides are (but not limited to) E. coli 0157 siderophore protein vaccines, E. coli 0157 type-3 secretory protein vaccines, Lyme disease (Borrelia burgdorfen outer surface protein A [OspA] and protein C [OspC] bactrins) vaccines, foot and mouth disease (FMD vaccines, inactivated virions or viral proteins), hemorrhagic septicemia (inactivated Pasteurella multocidia) vaccines, Johne's disease (inactivated mycobacterium avium subspecies paratuberculosis bactrin or protective immunogenic proteins) vaccines, Crohn's disease (inactivated mycobacterium avium subspecies paratuberculosis [MAP] bactrin or immunogenic proteins or viral vectors containing sequences coding for protective immunogenic proteins from [MAP]) vaccines, H1N1 vaccines, rabies vaccines.

### Example 11

### Co-Adjuvantation of Vaccines Containing ISCOM Based Adjuvants with Non-DNA Base-Containing Polynucleotides

The following example illustrates the use of the polynucleotide sequences of the present invention in enhancing the immune response to conventional vaccines containing ISCOMS (Immuno Stimulating Complexes) as adjuvant. A number of vaccines have been shown to benefit from being adjuvanted with ISCOMS. These vaccines are further adjuvanted using non-DNA base-containing polynucleotides of the present invention such that a stimulation of antibody production and protective activity is achieved. The dose of non-DNA base-containing polynucleotides used (within but not limited to the range 1-100 μg/vaccine dose) is added to the vaccine either during the manufacturing process during the formulation stage or immediately pre-immunization, and immunization of individuals is carried according to established vaccination procedures using established vaccine doses and routes of administration (conventionally but not limited to intramuscular injection). Immunization of individuals with ISCOM adjuvanted vaccines containing non-DNA base-containing polynucleotides results in a superior immune response when compared to vaccination with ISCOM adjuvanted vaccines alone, as determined by enhanced protective antibody titers, resistance to infectious organism exposure or elimination of established disease. One of skill in the art will recognize that a) benefit from the enhanced immune response is also achieved when the number of immunizations is not optimal (for example 1 or 2 immunizations instead of 3 or more immunizations) or when the dose of antigen(s) administered is less than optimal, as for example during pandemic immunization, and b) that antigen sparing in vaccines adjuvanted with ISCOMS will result from co-adjuvantation with non-DNA base-containing polynucleotides. Examples of vaccines that are adjuvanted with ISCOMS that benefit from the addition of non-DNA base-containing polynucleotides are (but not limited to) Rhodococcus equi vaccines, equine herpes virus type 2 (EHV-2) vaccines, contagious bovine pleuropneumonia (CPBB) vaccines, bovine virus diarrhea (BVDV) vaccines, Toxoplasma gondii vaccines, avian influenza virus vaccines, feline influenza virus vaccines, gonadotropic-releasing factor vaccines, Newcastle disease vaccines, respiratory syncytial viruses, herpes vaccines, measles virus vaccines, human papilloma vaccines.

### Example 12

### Co-Adjuvantation of Vaccines Containing Virosomes with Non-DNA Base-Containing Polynucleotides

The following example illustrates the use of the polynucleotide sequences of the present invention in enhancing the immune response to vaccines containing virosome. Virosomes represents reconstituted empty virus envelope devoid of nucleocapsid and the genetic material of the source virus. Virosomes are a market approved carrier and adjuvant system for the delivery of immunologically active substances. This predominantly synthetic carrier is broadly applicable with almost any antigen. The dose of non-DNA base-containing polynucleotides used (within but not limited to the range 1-100 μg/vaccine dose) is added to the vaccine either during the manufacturing process or immediately pre-immunization, and immunization of individuals is carried according to established vaccination procedures using established vaccine doses and routes of administration (conventionally intramuscular into the deltoid muscle). Immunization with virosomes containing non-DNA base-containing polynucleotides results in a superior immune response when compared to vaccination with virosome vaccines, as determined by enhanced protective antibody titers and resistance to infectious exposure or elimination of established disease. One of skill in the art will recognize that a) benefit from the enhanced immune response is also achieved when the number of immunizations is not optimal (for example 2 instead of 3 immunizations) or when the dose of antigen(s) administered is less than optimal, as for example during pandemic immunization, and b) that antigen sparing in vaccines adjuvanted with virosomes will result from co-adjuvantation with non-DNA base-containing polynucleotides. Examples of vaccines that benefit from the addition of non-DNA base-containing polynucleotides to virosomes are (but not limited to) Hepatitis A vaccines, Hepatitis A+B vaccines, HIV/AIDS vaccines, Malaria vaccines, Influenza vaccines and Respiratory Syncytial Virus (RSV) vaccines.

### Example 13

### Co-Adjuvantation of Vaccines Containing Monophosphoryl Lipid a with Non-DNA Base-Containing Polynucleotides

The following example illustrates the use of the polynucleotide sequences of the present invention in enhancing the immune response to vaccines containing an immune stimulant that is intended to stimulate the immune system, monophosphoryl lipid A (MPL). MPL is a Toll-like receptor-4 (TLR4) agonist that enhances antigen-specific immunity. Ragweed vaccine such as Pollinex Quattro® is intended to stimulate the production of immunoprotective (IgG) antibodies and is adjuvanted with MPL. The dose of non-DNA base-containing polynucleotides used (within but not limited to the range 1-100 μg/vaccine dose) is added to the vaccine either during the manufacturing process or immediately pre-immunization, and immunization of individuals is carried according to established vaccination procedures using established vaccine doses and routes of administration (conventionally intramuscular into the deltoid muscle). Immunization with MPL-containing vaccines containing non-DNA base-containing polynucleotides results in a superior immune response when compared to vaccination with MPL-containing vaccines, as determined by enhanced protective antibody titers and reduction of allergic episodes during ragweed season. One of skill in the art will recognize that a) benefit from the enhanced immune response is also achieved when the number of immunizations is not optimal (for example 2 instead of 4 immunizations). Examples of vaccines that benefit from the addition of non-DNA base-containing polynucleotides to MPL-containing vaccines are (but not limited to) ragweed vaccines, hepatitis B vaccines, Plasmodium falciparum vaccines, herpes (including genital herpes) virus vaccines, influenza vaccines, grass pollen allergy vaccines.

All patents, publications and abstracts cited above are incorporated herein by reference in their entirety. It should be understood that the foregoing relates only to preferred embodiments of the present invention and that numerous modifications or alterations may be made therein without departing from the spirit and the scope of the present invention as defined in the following claims.

We claim:

1. A composition comprising a synthetic non-DNA base-containing polynucleotide sequence of 3 to 30 bases in length, wherein the non-DNA base is one or more of nebularine, hypoxanthine or uracil, a pharmaceutically acceptable vehicle and one or more antigens.

2. The composition of claim 1, wherein the non-DNA base-containing polynucleotide sequence is 3 to 20 bases in length.

3. The composition of claim 1, wherein the non-DNA base-containing polynucleotide sequence is 3 to 9 bases in length.

4. The composition of claim 1, wherein the pharmaceutically acceptable vehicle comprises one or more adjuvant vehicles.

5. The composition of claim 4, wherein the one or more adjuvant vehicles is alum, an oil-based adjuvant, an immune stimulating complex, a virosome, or monophosphoryl lipid A, or an analog thereof.

6. The composition of claim 1, further comprising an immunomodulatory agent.

7. The composition of claim 1, wherein the synthetic non-DNA base-containing polynucleotide is any one of SEQ ID NOs: 1 to 30.

8. A composition comprising SEQ ID NO: 5.

9. The synthetic non-DNA base-containing polynucleotide sequence of claim 1, wherein the synthetic non-DNA base-containing polynucleotide sequence further comprises a TEG-cholesteryl moiety, one or more additional adenine, thymine, cytosine or guanine bases, or one or more additional nebularine, hypoxanthine or uracil bases, on the 5′ terminus or the 3′ terminus of the synthetic non-DNA base-containing polynucleotide sequence.

10. A method of modulating an immune response to one or more antigens comprising administering in vitro or in vivo a composition comprising a synthetic non-DNA base-containing polynucleotide sequence of 3 to 30 bases in length, wherein the non-DNA base is one or more of nebularine, hypoxanthine or uracil, a pharmaceutically acceptable vehicle and one or more antigens.

11. The method of claim 10, wherein the non-DNA base-containing polynucleotide sequence is 3 to 20 bases in length.

12. The method of claim 10, wherein the non-DNA base-containing polynucleotide sequence is 3 to 9 bases in length.

13. The method of claim 10, wherein the in vivo administration comprises administering to an animal or a human.

14. The method of claim 10, wherein the modulated immune response is a change in antibody response following a challenge with the antigen.

15. The method of claim 10, wherein modulating the immune response comprises stimulating an antigen presenting cell.

16. The method of claim 10, wherein modulating the immune response comprises enhancing an immune response to the antigen, wherein the amount of the antigen in the composition is less than the amount of the antigen required in the absence of the non-DNA base-containing polynucleotide to enhance the immune response.

17. The method of claim 10, wherein modulating the immune response comprises reducing the immune response to the antigen.

18. The method of claim 17, wherein the amount of the antigen in the composition is less than the amount of the antigen required in the absence of the non-DNA base-containing polynucleotide to reduce the immune response.

19. The method of claim 10, wherein the synthetic non-DNA base-containing polynucleotide is any one of SEQ ID NOs: 1 to 30.

20. The method of claim 10, wherein the pharmaceutically acceptable vehicle comprises one or more adjuvant vehicles.

21. The method of claim 10, wherein the one or more adjuvant vehicles is alum, an oil-based adjuvant, an immune stimulating complex, a virosome, or monophosphoryl lipid A, or an analog thereof.

22. The method of claim 10, further comprising administering an immunomodulatory agent.

23. The method of claim 10, wherein the synthetic non-DNA base-containing polynucleotide sequence further comprises a TEG-cholesteryl moiety, one or more additional adenine, thymine, cytosine or guanine bases, or one or more additional nebularine, hypoxanthine or uracil bases, on the 5′ terminus or the 3′ terminus of the synthetic non-DNA base-containing polynucleotide sequence.
